(19) United States
(12) Patent Application Publication
(10) Pub. No.: US 2024/0082505 A1
(43) Pub. Date: Mar. 14, 2024

(54) A CASSETTE UNIT SUB-ASSEMBLY FOR A MEDICAMENT DELIVERY DEVICE

(71) Applicant: SHL Medical AG, Zug (CH)

(72) Inventors: Ming-Ting YIN, Taoyuan City (TW); Oscar ALEXANDERSSON, Nacka Strand (SE); Daniel CARLSSON, Nacka Strand (SE); Johan ZANDER, Nacka Strand (SE)

(21) Appl. No.: 18/556,734

(22) PCT Filed: Apr. 19, 2022
(86) PCT No.: PCT/EP2022/060242

§ 371 Date: Oct. 23, 2023

Related U.S. Application Data
(60) Provisional application No. 63/250,313, filed on Sep. 30, 2021.
(60) Provisional application No. 63/232,753, filed on Aug. 13, 2021.
(60) Provisional application No. 63/210,524, filed on Jun. 15, 2021.

(30) Foreign Application Priority Data
Apr. 28, 2021 (EP) 21171037.1

(51) Int. Cl. A61M 5/32

(52) U.S. Cl. CPC A61M 5/322; A61M 5/326

(57) ABSTRACT

A cassette unit sub-assembly for a medicament delivery device, the sub-assembly comprising: a body extending along a longitudinal axis from a proximal end to a distal end; a delivery member guard coaxially attached to the body and being axially movable relative to the body along the longitudinal axis; a lock member attached to the body; the lock member is movable relative to the delivery member guard between a locked position and an unlocked position; the lock member comprises a distally directed surface adjacent to a proximally directed surface of the delivery member guard in the locked position; and the distally directed surface of the lock member is spaced apart in a circumferential direction relative to the longitudinal axis from the proximally directed surface of the delivery member guard in the unlocked position.

### TECHNICAL FIELD

The present disclosure generally relates to cassette unit sub-assemblies for medicament delivery devices such as reusable autoinjectors or injectors with two detachable parts, and particularly to sub-assemblies for locking delivery member guards of such units before use.

### BACKGROUND

Medicament delivery devices such as pen type manual injectors or auto-injectors are generally known for the self-administration of a medicament by patients without formal medical training. For example, patients suffering from diabetes may require repeated injections of insulin, or patients may require regular injections of other types of medicaments, such as a growth hormone.

Some medicament delivery devices have two parts that are interconnected during use. One part often receives a medicament container that contains a medicament, such as a cartridge or a syringe; this part usually can be called a cassette or a cassette unit. The other part often comprises a driver for actuating expulsion of the medicament.

Medicament delivery devices designed in this way are commonly reusable. The cassette is a disposable part, and the other part with the driver usually is a reusable part. The driver may comprise a power unit such as a spring or a motor and thus can be re-used for a number of medicament deliveries.

Medicament delivery devices are commonly designed to comprise a delivery member guard for protecting a medicament delivery member (such as a needle or a spray nozzle) for delivering the medicament to the end-user from contamination; and for protecting the end-user from being injured by the medicament delivery member.

It is common to find a locking mechanism/device for locking the delivery member guard to prevent a distal movement of the delivery member guard after use, usually locking the delivery member guard in the final, used position. Before use, the delivery member guard is often protected by a cap to stop the user from accidentally pushing the delivery member guard and exposing the medicament delivery member. It has been appreciated, however, that alternative solutions for medicament delivery member protection before use could be advantageous.

### SUMMARY

The invention is defined by the appended claims, to which reference should now be made. In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a cassette unit sub-assembly for a medicament delivery device, the sub-assembly comprising: a body extending along a longitudinal axis from a proximal end to a distal end; a delivery member guard coaxially attached to the body and being axially movable relative to the body along the longitudinal axis; a lock member attached to the body; the lock member is movable relative to the delivery member guard between a locked position and an unlocked position; the lock member comprises a distally directed surface adjacent to a proximally directed surface of the delivery member guard in the locked position; and the distally directed surface of the lock member is spaced apart in a circumferential direction relative to the longitudinal axis from the proximally directed surface of the delivery member guard in the unlocked position.

The lock member is configured to lock the delivery member guard from moving in the proximal direction relative to the body when the lock member is in the locked position, and to unlock the delivery member guard so that the delivery member guard can be freely moved in the proximal direction relative to the body.

The sub-assembly provided by this invention therefore can releasably prevent the medicament delivery guard from moving relative to the body by positioning the lock member in the locked position or the unlocked position.

The proximal movement of the delivery member guard can therefore be used to trigger another action of the medicament delivery device, e.g. priming, needle mounting, enabling an end-user to mount the medicament delivery member, or in some cases as a second safety design. For example, there may be a safety mechanism of the delivery member guard on the cassette unit with the delivery member guard and the medicament delivery member. The safety mechanism is designed to prevent the delivery member guard from moving in the distal position before use until the cassette unit of the medicament delivery device has been properly assembled to the other part of the medicament delivery device, so the end-user will not accidentally get an injury or contaminate the delivery member. However, when the end-user assembles two parts of the medicament delivery device together, sometimes the end-user may press on the delivery member guard in the distal direction; and once the safety mechanism has been released, the end-user may contact the delivery member because the delivery member guard just suddenly moves in the distal direction. In such case, the proximal movement of the delivery member guard can be a second safety design; for example, the delivery member guard cannot move in the distal direction unless the delivery member guard moves into a proximal position first; or the delivery member guard cannot move in the distal direction with a long enough distance to reveal the medicament deliver member. Therefore, the medicament delivery member will not be revealed to the end-user; unless (i) the cassette unit has been properly assembled to the other part of the medicament delivery device; and (ii) the end-user is not pressing in the distal direction on the delivery member guard (so the delivery member guard cannot move into the proximal “free to move” position).

Preferably, according to another embodiment, the lock member is rotatable relative to the delivery member guard around the longitudinal axis between the locked position and the unlocked position.

Alternatively, according to another embodiment, the lock member is linearly slidable relative to the delivery member guard in the direction transverse to the longitudinal axis from the locked position to the unlocked position.

Preferably, according to another embodiment, the lock member is a removable pin that is configured to be removed from the delivery member guard with a linear slide relative to the delivery member guard in the direction transverse to the longitudinal axis.

Alternatively, according to another embodiment, the lock member is radially flexible relative to the longitudinal axis and to the delivery member guard from the locked position to the unlocked position.

Preferably, according to another embodiment, the lock member is a clip radially flexible relative to the delivery member guard from the locked position to the unlocked position.

Preferably, according to another embodiment, the lock member is a removable clip that is configured to be removed from the delivery member guard when the removable clip flexes from the locked position to the unlocked position.

Preferably, according to another embodiment, the cassette unit sub-assembly comprises a biasing member arranged between a first distally directed surface of the delivery member guard and a proximally directed surface of the body.

According to another embodiment, the delivery member guard comprises a ledge radially extending relative to the longitudinal axis; and the first distally directed surface of the delivery member guard is defined by a part of the ledge.

Preferably, according to another embodiment, the delivery member guard comprises a protrusion radially extending relative to the longitudinal axis; and the proximally directed surface of the delivery member guard is a part of the protrusion.

Preferably, according to another embodiment, the protrusion extends radially away from the ledge.

Preferably, according to another embodiment, the lock member comprises a tubular body enclosing a portion of the delivery member guard.

According to another embodiment, the lock member comprises a ledge extending in a circumferential direction relative to the longitudinal axis along a portion of an inner surface of the tubular body of the lock member; and the distally directed surface of the lock member is a part of the ledge.

Alternatively, according to another embodiment, the lock member comprises a recess extending in a circumferential direction relative to the longitudinal axis along a portion of an inner surface of the tubular body of the lock member; the recess radially faces toward the longitudinal axis; and the distally directed surface of the lock member is a part of the recess.

Preferably, according to another embodiment, the cassette unit sub-assembly comprises an actuator attached to the body and configured to move relative to the body in the direction of the longitudinal axis between a proximal position and a distal position; the actuator comprises an elongated body; and the actuator comprises an interaction part extending toward the distal end of the body from the elongated body of the actuator.

Preferably, according to another embodiment, the delivery member guard is movable relative to the body between a distal trigger position and a proximal after-use position; and the delivery member guard comprises a second distally directed surface.

According to another embodiment, the actuator comprises a proximally directed lock-out surface; and the second distally directed surface of the delivery member guard is adjacent to the proximally directed lock-out surface of the actuator when the delivery member guard is in the proximal after-use position.

Alternatively, according to another embodiment, the cassette unit sub-assembly comprises an inner housing arranged within the delivery member guard and the actuator; and the inner housing comprises a proximally directed lock-out surface adjacent to the second distally directed surface of the delivery member guard when the delivery member guard is in the proximal after-use position.

According to another embodiment, the actuator is positioned between the proximally directed lock-out surface of the inner housing and the second distally directed surface of the delivery member guard in a direction transverse to the longitudinal axis when the delivery member guard is not in the proximal after-use position.

Preferably, according to another embodiment, the actuator comprises a proximally directed retaining surface adjacent to a distally directed surface of the body when the actuator is axially moved to the distal position from the proximal position.

Preferably, according to another embodiment, the interaction part of the actuator protrudes from the distal end of the body when the actuator is in the distal position.

According to another embodiment, the cassette unit sub-assembly of the invention can be used in a cassette unit for a medicament delivery device. The cassette comprising: a delivery member cover assembly fixed to the proximal end of the body and being rotationally releasable relative to the body; the delivery member cover assembly comprises: a tubular outer cap; an inner cap at least partially surrounded by the tubular outer cap; a clutch member rotationally fixed to the inner cap and positioned longitudinally between the tubular outer cap and the delivery member guard; the clutch member comprises a clutch biasing member, the clutch member being axially movable relative to the outer cap between a relaxed position and a tensioned position; the tubular outer cap is axially fixed with and rotatable relative to the inner cap; the tubular outer cap comprises a plurality of engaging members and the clutch member comprises a plurality of counter engaging members; and the plurality of engaging members is adjacent with the plurality of counter engaging members when the clutch biasing member is in the tensioned position.

Preferably, according to another embodiment, the plurality of engaging members of the tubular outer cap is a plurality of protrusions; the plurality of protrusions extends radially inwardly relative to the longitudinal axis from an inner surface of the tubular outer cap.

According to another embodiment, the plurality of counter engaging members of the clutch member is a plurality of counter protrusions; the plurality of counter protrusions extends radially outwardly relative to the longitudinal axis from an outer surface of the inner cap.

Alternatively, according to another embodiment, the plurality of counter engaging members of the clutch member is a plurality of slots; the plurality of slots faces radially outwardly relative to the longitudinal axis from an outer surface of the inner cap.

Preferably, according to another embodiment, the clutch member comprises a proximal connector and a distal connector; the clutch biasing member is longitudinally positioned between the proximal connector and the distal connector; and the distal connector is adjacent to a proximal end of the delivery member guard and the proximal connector is releasably adjacent to a distally directed surface on the inner surface of the tubular outer cap.

According to another embodiment, the plurality of counter engaging members of the clutch member is arranged on an outer surface of the distal connector.

Alternatively, according to another embodiment, the plurality of counter engaging members of the clutch member is arranged on an outer surface of the proximal connector.

According to another embodiment, the plurality of counter engaging members comprises a flexible arm extending partially around the longitudinal axis.

Preferably, according to another embodiment, the body comprises a bayonet connector on an outer surface of the body for attachment to a part of a medicament delivery device.

Preferably, according to another embodiment, the body comprises a snap-fit connector on an outer surface of the body for attachment to a part of a medicament delivery device.

Preferably, according to another embodiment, the lock member comprises a flange; the flange protrudes from the outer surface of the body, and the flange is aligned with the bayonet connector of the body in a longitudinal direction.

Another embodiment of the invention provides a cassette unit sub-assembly for a medicament delivery device, the cassette unit sub-assembly comprising: a body extending along a longitudinal axis from a proximal end to a distal end; a delivery member guard coaxially attached to the body and being proximally biased to axially move relative to the body from a distal position to a proximal position; and a lock member attached to the body; the lock member is movable relative to the delivery member guard between a locked position and an unlocked position; and the lock member is configured to lock the delivery member guard in the distal position when the lock member is in the locked position.

Preferably, according to another embodiment, the lock member is rotatable relative to the delivery member guard around the longitudinal axis between a locked position and an unlocked position.

Preferably, according to another embodiment, the lock member comprises a distally directed surface facing a proximally directed surface of the delivery member guard in the locked position; and the distally directed surface of the lock member is apart from the proximally directed surface of the delivery member guard in the unlocked position.

Preferably, according to another embodiment, the delivery member guard comprises a protrusion radially extending relative to the longitudinal axis; and the proximally directed surface of the delivery member guard is a part of the protrusion.

Preferably, according to another embodiment, an actuator configured to be moved by the delivery member guard along the longitudinal axis relative to the body.

Preferably, according to another embodiment, the actuator comprises an interaction part configured to interact with a part of the medicament delivery device comprising the cassette unit with the cassette unit sub-assembly.

Preferably, according to another embodiment, the delivery member guard is movable relative to the body between distal trigger position and a proximal after-use position; and the actuator is configured to lock delivery member guard in the proximal after-use position when the delivery member guard moves to the proximal after-use position from the distal trigger position.

Preferably, according to another embodiment, the cassette unit sub-assembly of this invention can be used with a cassette unit for a medicament delivery device; besides the cassette unit, the medicament delivery device comprises a drive unit, and the cassette unit is releasably attached to the medicament delivery device drive unit.

Preferably, according to another embodiment, the cassette unit of this invention can be used with a medicament delivery device comprising a reusable drive assembly; the reusable drive assembly comprises a tubular housing extending along the longitudinal axis from a proximal end and a distal end; and the cassette unit is releasably attached to the reusable drive assembly through a bayonet connection on an inner surface on the proximal end of the reusable drive assembly and on an outer surface on the distal end of the body of the cassette unit.

According to another embodiment, the inner surface on the proximal end of the reusable drive assembly comprises a bayonet groove; and wherein the outer surface on the distal end of the body of the cassette unit comprises a bayonet protrusion.

Another aspect of the invention provides a cassette unit sub-assembly for a medicament delivery device, the sub-assembly comprising: a housing extending along a longitudinal axis between a proximal end and a distal end; a delivery member guard telescopically arranged to the proximal end of the housing; and an actuator axially movable and rotatable relative to the delivery member guard; the delivery member guard comprises an interaction part configured to rotate and axially move the actuator relative to the housing through interacting with a chamfer arranged on the actuator.

Preferably, according to another embodiment, the interaction part is a protrusion or a distally directed arm.

Preferably, according to another embodiment, the actuator comprises an actuator body, and a track arranged on a wall of the actuator body.

Preferably, according to another embodiment, the track comprises a first track extending in the direction of the longitudinal axis and a second track comprising the chamfer.

Preferably, according to another embodiment, the track is formed by a cut-out, or a recess, or multiple transverse extending ledges arranged on the body of the actuator.

Preferably, according to another embodiment, the medicament delivery device is an injection device, an inhalation device, or a medical sprayer.

Preferably, according to another embodiment, the medicament delivery device cassette unit is for accommodating a medicament container.

Preferably, according to another embodiment, the drive unit comprises a driver for actuating the medicament contained in the medicament container accommodated by the cassette unit.

Preferably, according to another embodiment, the sub-assembly comprises a distal lid attachable to the distal end of the body.

Preferably, according to another embodiment, the distal lid is a part of the body of the sub-assembly.

Preferably, according to another embodiment, the distal lid comprises a lid body formed in a shape in accordance with the shaped of the medicament container received within the body.

Preferably, according to another embodiment, the body comprises a tubular portion.

Preferably, according to another embodiment, the distal lid comprising a counter inclined surface configured to interact with an inclined surface of the actuator when the actuator is axially moved by the delivery member guard.

Preferably, according to another embodiment, the second track comprises a lockout portion, the protrusion of the delivery member guard is configured to move into the lockout portion together with the distal movement of the delivery member guard towards a proximal after-use position of the delivery member guard.

Preferably, according to another embodiment, the protrusion is arranged at a flexible portion of the delivery member guard or a distal frame of the lockout portion of the actuator is flexible.

Preferably, according to another embodiment, the actuator comprises a locking tab configured to move into a locking cut-out of the distal lid together with the distal movement of the delivery member guard towards the proximal after-use position of the delivery member guard.

Preferably, according to another embodiment, the distal lid comprises a container support for supporting the medicament container received within the body; the container support comprises a proximally directed surface.

Preferably, according to another embodiment, the container support is a flexible arm.

Preferably, according to another embodiment, the flexible arm of the container support can be formed by a cut-out, opening towards the direction transverse to the longitudinal axis, on the lid body.

Preferably, according to another embodiment, the flexible arm may extend from the lid body towards the distal direction.

Preferably, according to another embodiment, the proximally directed surface of the container support is formed on an inner surface of the flexible arm or formed on a distal tip of the flexible arm.

Preferably, according to another embodiment, the container support is configured to prevent an axial movement of the medicament container received within the body in the distal direction and/or be used to tolerate the received medicament container with different dimension, e.g. certain length and/or width of the received medicament container, regarding to the engineering tolerance.

Preferably, according to another embodiment, the medicament delivery device with the sub-assembly as this invention can be operated by a method comprising the step of connecting the cassette unit to the drive unit.

Preferably, according to another embodiment, the method comprises the step of connecting the cassette unit to the drive unit by rotating the cassette unit relative to the drive unit around the longitudinal axis.

Preferably, according to another embodiment, the method comprises the step of rotating the lock member relative to body from the locked position to the unlocked position; moving the delivery member guard along the longitudinal axis relative to the body in a proximal direction; compressing the clutch biasing member from the relaxed position to the tensioned position by the proximal movement of the delivery member guard; and rotating the outer cap together with the inner cap around the longitudinal axis relative to the body to remove the delivery member cover assembly from the body through an engagement between the plurality engaging members and the plurality counter engaging members.

Preferably, according to another embodiment, the method comprises the step of inserting the distal end of the body of the cassette unit into the proximal end of the housing of the drive assembly; rotating the cassette unit relative to the housing of the drive assembly along the bayonet connection; and rotating the body of the cassette unit relative to the lock member around the longitudinal axis while rotating the cassette unit relative to the housing of the drive assembly along the bayonet connection.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a perspective view of a medicament delivery device with a cassette unit and a medicament delivery device drive unit.

FIG. 2 schematically shows an exploded view of a sub-assembly for a cassette unit of the medicament delivery device of FIG. 1.

FIG. 3 schematically shows a perspective view of a lock member of the sub-assembly of FIG. 2 and a body of the sub-assembly of FIG. 2.

FIGS. 4A-4B schematically show perspective views of the lock member of FIG. 3 in different embodiments.

FIG. 5 schematically shows a perspective view of a delivery member guard of the sub-assembly of FIG. 2.

FIGS. 6A-6E schematically show distal views of the delivery member guard of the sub-assembly of FIG. 2 in two examples and the lock member of the sub-assembly of FIG. 2, when the lock member is in a locked position and an unlocked position.

FIGS. 6F-6G schematically show perspective views of an actuator of the sub-assembly of FIG. 2 and a distal lid of the sub-assembly of FIG. 2.

FIG. 7 schematically shows a perspective view of a distal lid of the sub-assembly of FIG. 2 in another example, and the body of the sub-assembly of FIG. 2.

FIG. 8 schematically shows a perspective view of the distal lid of FIG. 7, a distal portion of the delivery member guard of the sub-assembly of FIG. 2 and a biasing member of the sub-assembly of FIG. 2.

FIG. 9 schematically shows a perspective view of the delivery member guard of the sub-assembly of FIG. 2 and an actuator of the sub-assembly of FIG. 2.

FIG. 10 schematically shows a perspective view of the delivery member guard of FIG. 9 and the actuator of FIG. 9.

FIGS. 11A-11B schematically show cross-section views of the delivery member guard of FIG. 9 and the actuator of FIG. 9.

FIGS. 12A-12B schematically show cross-section views of the delivery member guard of FIG. 9 and the actuator of FIG. 9 when they are assembled with the distal lid of FIG. 7 and the biasing member of FIG. 8.

FIG. 13 schematically shows a perspective view of the distal lid of FIG. 7.

FIGS. 14A-14B schematically show side views of the distal lid of FIG. 7 and the actuator of FIG. 9.

FIGS. 15A-15B schematically show side views of the delivery member guard of FIG. 5, the distal lid of FIG. 7 and the actuator of FIG. 9.

FIG. 16 schematically shows perspective view of the actuator of the sub-assembly of FIG. 2 in another example.

FIG. 17 schematically shows perspective view of the deliver member guard of FIG. 5 and an inner housing of the sub-assembly of FIG. 2.

FIG. 18A schematically shows a perspective view of the inner housing of FIG. 17 the delivery member guard of FIG. 5 and the actuator of FIG. 16.

FIG. 18B schematically shows a cross-section view of FIG. 18A.

FIG. 18C schematically shows a perspective view of the inner housing of FIG. 17 the delivery member guard of FIG. 5 and the actuator of FIG. 16.

FIGS. 19A-19B schematically show perspective views of the inner housing of FIG. 17 the delivery member guard of FIG. 5 and the actuator of FIG. 16.

FIGS. 19C-19J schematically show an operation sequence of an interaction between the actuator of FIG. 6F, the distal lid of FIG. 6G, and the delivery member guard of FIG. 6C.

FIG. 20 schematically shows a side view of the cassette unit with a medicament delivery member set.

FIG. 21 schematically shows an exploded view of the sub-assembly of FIG. 2 with the medicament delivery member set of FIG. 20.

FIGS. 22A-22B schematically show perspective views of the medicament delivery member set of FIG. 20.

FIG. 23 schematically shows a perspective view of an inner cap of the medicament delivery member set of FIG. 20.

FIG. 24 schematically shows a distal view of a clutch member of the medicament delivery member set of FIG. 20.

FIGS. 25A-25B schematically show perspective views of the medicament delivery member set of FIG. 20.

FIGS. 26A-26B schematically show perspective views of the outer and inner cap of the medicament delivery member set in another example.

### DETAILED DESCRIPTION

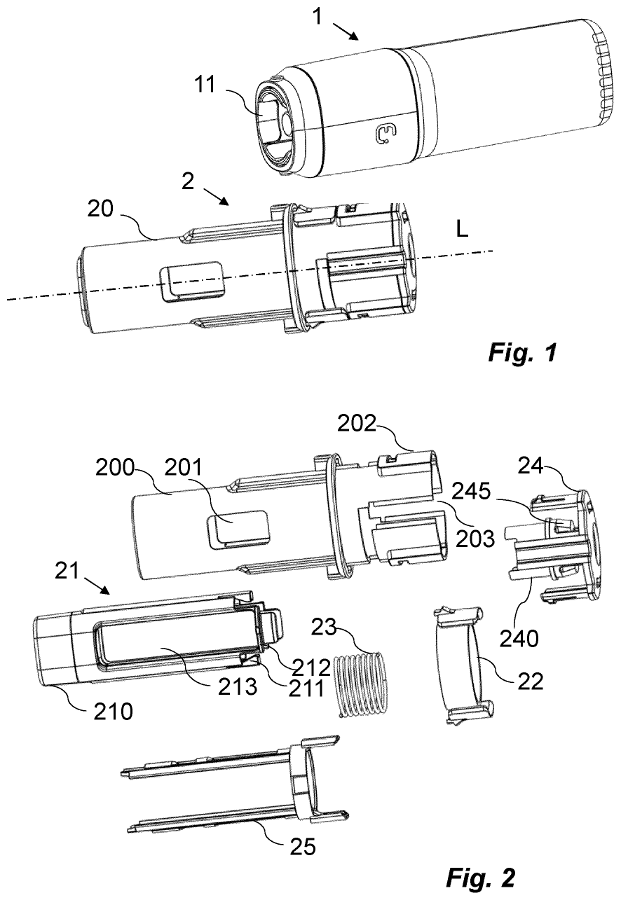

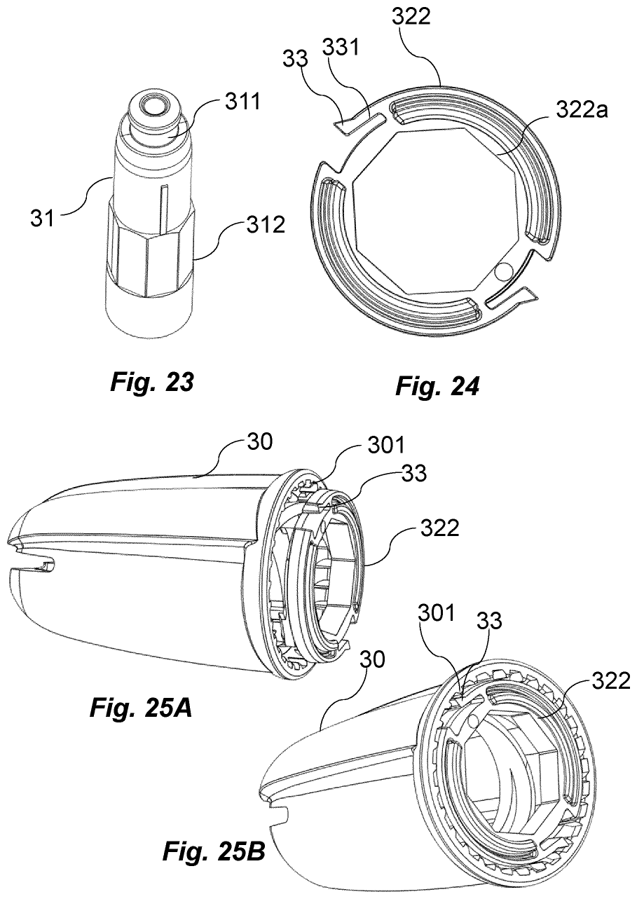

FIGS. 1-36D illustrate a cassette unit sub-assembly for a medicament delivery device. The term “cassette unit sub-assembly” will sometimes be abbreviated as “sub-assembly” in this description. It should be noted that both the term “cassette unit sub-assembly” and the term “sub-assembly” are used with the same meaning in this description. The cassette unit is usually used with a medicament delivery device with two detachable parts, as shown in FIG. 1. The first part of the medicament delivery device, or the cassette unit 2, is usually for accommodating a medicament container, e.g. a syringe or cartridge, with a medicament contained within. The syringe or cartridge can be made of glass or plastic. The syringe or cartridge may comprise a rubber stopper sealing on a distal end of the syringe or cartridge, and a septum or medicament delivery member with a rubber sheath sealing on a proximal end of the syringe or cartridge. Alternatively, the syringe or cartridge may be collapsible with only a septum or medicament delivery member with a rubber delivery member sheath sealing a proximal end of the syringe or cartridge.

The second part of the medicament delivery device, or a drive unit 1, usually comprises a driver for actuating the medicament contained within the medicament container in the proximal direction of the medicament delivery device, expelled to an end-user. The drive unit 1 may comprise a plunger rod for pushing on the medicament container. In one example, the stopper of the medicament container can therefore be moved in the proximal direction relative to the other part of the medicament container. In another example, the plunger rod may collapse the medicament container by pushing the medicament container in the proximal direction relative to the cassette unit 2. The drive unit 1 also comprises a power source, such as a spring, a gas canister, or a motor-driven gear set, for moving the plunger rod in the proximal direction of the medicament delivery device. The focus of the concept described herein is a cassette unit sub-assembly for the medicament delivery device, not the drive unit, and various different types of drive units could be used with the cassette units with the sub-assembly described herein. As such, the drive unit will not be comprehensively described.

The sub-assembly of the invention comprises a body 20; 20′ extending along a longitudinal axis L from a proximal end to a distal end; a delivery member guard 21; 21′; 21″ coaxially attached to the body and being axially movable relative to the body 20; 20′ along the longitudinal axis; a lock member 22; 22′; 22″ attached to the body 20; 20′. The lock member 22; 22′; 22″ is movable relative to the delivery member guard 21; 21′; 21″ between a locked position and an unlocked position. The lock member 22; 22′; 22″ comprises a distally directed surface adjacent to a proximally directed surface of the delivery member guard 21; 21′; 21″ in the locked position; the distally directed surface of the lock member 22; 22′; 22″ is spaced apart in a circumferential direction relative to the longitudinal axis L from the proximally directed surface of the delivery member guard 21; 21′; 21″ in the unlocked position.

In a preferred embodiment, the body 20; 20′ may be formed as a generally tubular-shaped component, which is a shape corresponding to most common medicament containers, so that the sub-assembly can keep the medicament delivery device compact. However, the body can be formed in any other suitable shape that is able to contain the medicament container.

As shown in FIGS. 1-2, the body 20 is configured to accommodate the medicament container in an outer shell 200. If the visibility of the medicament container is required, the outer shell 200 may comprise a window 201 aligned with the medicament container, so that the end-user can observe the medicament through the window 201. On the other hand, the outer shell 200 may be transparent, so that the body does not need the window. The cassette unit 2 is configured to be releasably attached to the drive unit 1 through a releasable connection between the body 20 and the drive unit when the end-user plans to carry out a medicament delivery operation. The body 20 comprises a connector configured to connect with a counter connector on the drive unit 1. In one example, the connector and the counter connector can form a bayonet connection, meaning that one of the body 20 and the drive unit 1 comprises a bayonet protrusion, and the other one of the body 20 and the drive unit 1 comprises a bayonet recess/cut-out. For example, the outer body 20 may comprise a bayonet connector 202 on the outer shell 200 of the body 20, such as a bayonet protrusion as shown in FIG. 2; and the drive unit 1 comprises a counter bayonet connector 11, such as a bayonet recess as shown in FIG. 1. In this example, the body 20 is configured to be axially inserted, along the longitudinal axis L, into a proximal part of the drive unit 1, through the bayonet connector 202 and the counter bayonet connector 11; then the body 20 needs to be rotated relative to the drive unit 1, around the longitudinal axis L, so that the cassette unit 2 is properly attached to the drive unit.

Alternatively, the connection between the body 20 and the drive unit 1 can be another suitable connection, such as a snap-fit connection or thread connection; or the cassette unit may be completely received into the drive unit; in this example, the portion of the drive unit for receiving the cassette unit may be a form-fit connection to releasably receive the cassette unit. In the case that the cassette unit is completely received into the drive unit, the shape of the body may be dependent on the shape of the portion of the drive unit for receiving the cassette unit.

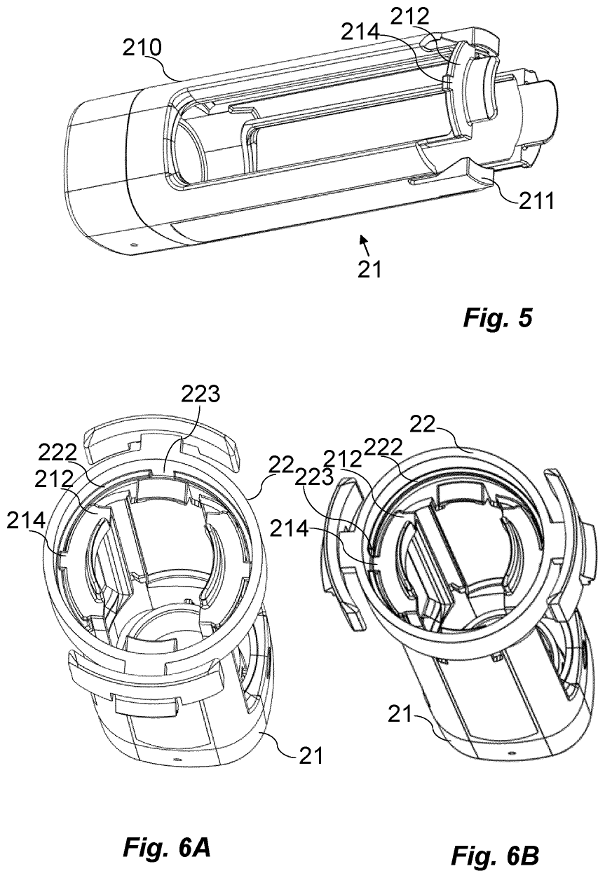

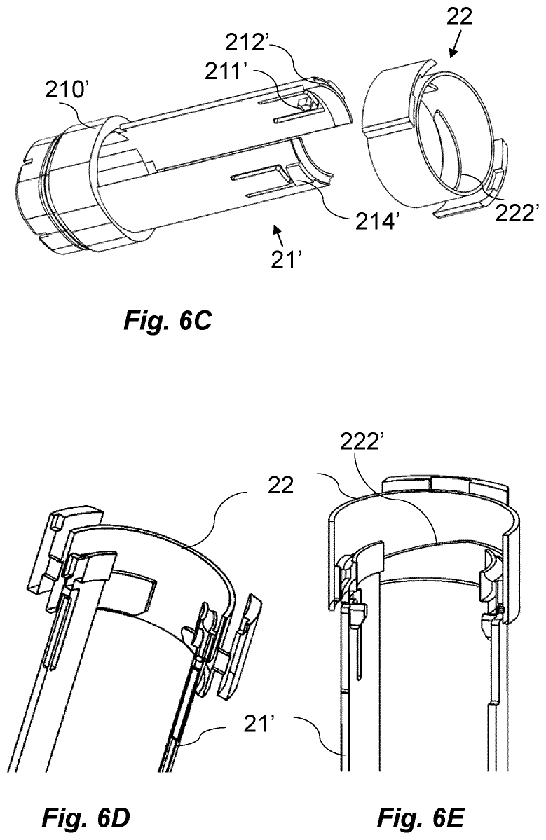

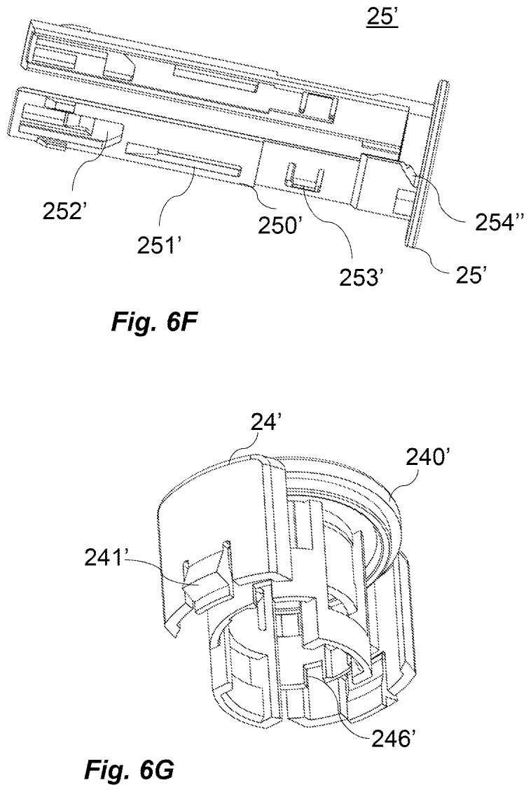

The sub-assembly of the invention comprises the delivery member guard 21; 21′, as shown in FIG. 2, FIG. 5, and FIG. 6C. The delivery member guard 21; 21′ comprises a front shield 210; 210′ configured to protrude from the proximal end of the body 20. The front shield 210; 201′ is configured to shield a medicament delivery member integral with or being attachable to the medicament container when the medicament delivery device is used by the end-user. In a preferred example, the front shield 210; 210′ is a tubular-shaped element with an opening in the proximal end of the front shield 210; 210′, so that the front shield 210; 210′ of the delivery member guard 21; 21′ is coaxially attached to the body 20 and is axially movable relative to the body 20 along the longitudinal axis L between a distal position and a proximal position. Similarly, the front shield 210; 210′ of the delivery member guard is a similar shape to the body 20 to make the device compact, but the delivery member guard can also be formed in any other suitable shape which can axially movable relative to the body 20 along the longitudinal axis L.

The delivery member guard 21 comprises an arm 211 extending from a distal end of the front shield 210 towards the distal direction of the body 20 of the sub-assembly. Preferably, the delivery member guard 21 comprises a protrusion 214, as shown in FIG. 5, radially extending relative to the longitudinal axis L. The delivery member guard 21 optionally comprises a ledge 212 arranged on the distal end of the delivery member guard. In a preferred example, the protrusion 214 extends radially relative to the longitudinal axis L away from the ledge 212. The delivery member guard 21 optionally comprises a window portion 213 configured to be aligned with the window 201 on the outer shell 200 of the body 20. The window portion 213 can be an aperture or a portion formed by a transparent material.

In a preferred example, the delivery member guard 21 is partially arranged within the body 20, namely the delivery member guard 21 is partially arranged within the outer shell 200 of the body 20. In this example, preferably, the arm 211 and the ledge 212 are fully arranged within the body 20; and the front shield 210 is fully or partially protruding from the proximal end of the body 20 so that the front shield 210 is able to cover the medicament delivery member. In one example, the arm 211 of the delivery member guard 21 is configured to interact with a part of the drive unit 1 of the medicament delivery device comprises the sub-assembly of the invention. For example, once the cassette unit 2 that comprises the sub-assembly has been assembled to the drive unit 1, a further distal movement of the delivery member guard 21 may move the arm 211 into the drive unit 1 for releasing a plunger rod connected with a power source, e.g. a spring, a gas canister, a motor; or switching on the power source.

The sub-assembly comprises the lock member 22, as shown in FIGS. 2-4B. The lock member 22 is attached to the body 20. The lock member 22 can be removably or irremovably attached to the body 20. The lock member 22 comprises a body 220. In a preferred example, the body 220 of the lock member 22 is a tubular body 220. The lock member 22 is movable relative to the body 20 in the locked position and the unlocked position. As shown in FIGS. 4A-B, the lock member 22 comprises a distally directed surface adjacent to a proximally directed surface of the delivery member guard 21; 21′ in the locked position; and the distally directed surface of the lock member 22 is spaced apart from the proximally directed surface of the delivery member guard 21; 21′ in the unlocked position. In a preferred example, the proximally directed surface of the delivery member guard 21; 21′ is defined by the protrusion 214.

The sub-assembly of the invention is arranged so that when the lock member 22; 22′; 22″ is in the locked position, the delivery member guard 21; 21′; 21″ is locked in the distal position; and when the lock member 22; 22′; 22″ is in the unlocked position, the delivery member guard 21; 21′; 21″ is released from the distal position; so that if the delivery member guard 21; 21′; 21″ is subjected to a proximal force, the delivery member guard 21; 21′; 21″ can move into the proximal position.

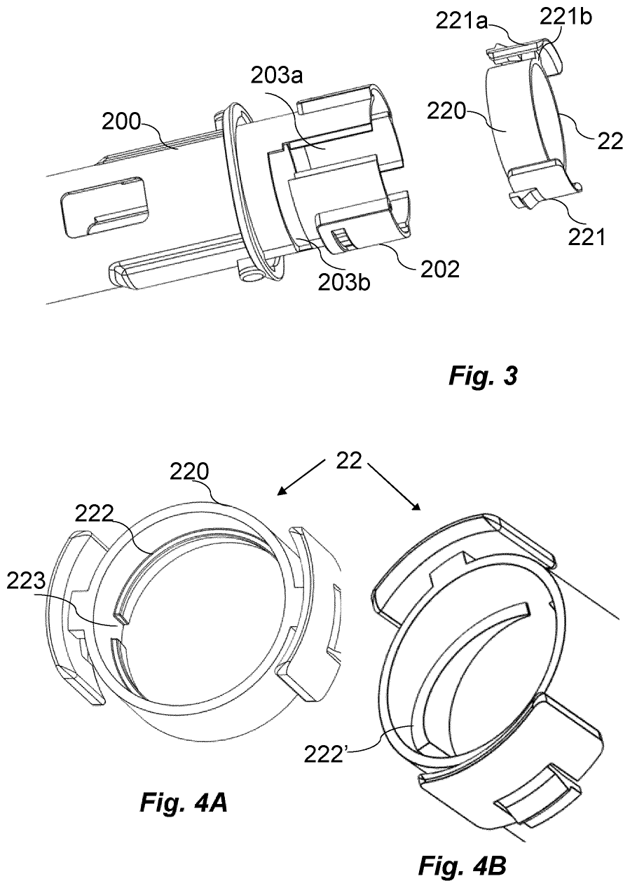

In one example, the lock member 22; 22′; 22″ is rotatably attached to the body 20; 20′ between the locked position and the unlocked position. In this example, for the purpose of attaching the lock member 22 to the body 20, the lock member 22 comprises a flange 221 as shown in FIG. 3. In this example, as shown in FIG. 3, the flange 221 comprises an enlarged part 221a and a transverse part 221b. The transverse part 221b extends between the enlarged part 221a and the body 220 of the lock member 22. In this example, the body 20 comprises an assembly track 203 formed by a cut-out/recess on the distal end of the body 20. Preferably on the distal portion of the outer shell 200 of the body 20, as shown in FIG. 3. The assembly track 203 comprises a longitudinal part 203a and a transverse part 203b. The longitudinal part 203a is a longitudinal cut-out/recess on the distal end of the body 20, and the transverse part 203b is a transverse cut-out/recess on the distal end of the body 20. The longitudinal part 203a of the assembly track 203 comprises a distal end and a proximal end. The transverse part 203b of the assembly track 203 comprises a first circumferential end and a second circumferential end. The proximal end of the longitudinal part 203a is connected to the first circumferential end of the transverse part 203b of the assembly track 203.

A dimension of the longitudinal part 203a and the transverse part 203b, observed from a direction transverse to the longitudinal axis L, is greater than a dimension of the transverse part 221b of the flange 221 of the lock member 22, but smaller than a dimension of the enlarged part 221a of the flange 221 of the lock member 22.

During assembly, the lock member 22 is configured to be attached to the body 20 through the flange 221 of the lock member 22 and the assembly track 203 of the body 20. The transverse part 221b of the flange 221 of the lock member 22 is aimed at the distal end of the longitudinal part 203a of the assembly track 203; inserted into the longitudinal part 203a through the distal end of the longitudinal part 203a; moved along the longitudinal part 203a of the assembly track 203 from the distal end of the longitudinal part 203a to the proximal end of the longitudinal part; entered into the transverse part 203b of the assembly track 203 from the first circumferential end of the transverse part 203b, and moved along the transverse part 203b of the assembly track 203 until being blocked by the second circumferential end of the transverse part 203b.

The body 220 of the lock member 22 is dimensioned to fit with the dimension of the distal end of the outer shell 200, so that the body 220 of the lock member 22 can be snugly received within the distal end of the outer shell 200 after assembly. Alternatively, the dimension of the longitudinal part 203a and the transverse part 203b is smaller than a dimension of the enlarged part 221a of the flange 221 of the lock member 22, so a transverse movement of the lock member 22 relative to the body 20 is blocked by the enlarged part 221a.

In a preferred example, the enlarged part 221a of the flange 221 protrudes from the outer surface of the distal end of the outer shell 200 of the body 20. In one example the bayonet connector 202 of the body 20 is a bayonet protrusion. The flange 22, specifically the enlarged part 221a of the flange 221, is aligned with the bayonet connector 202 of the body 20 in a longitudinal direction, as shown in FIG. 3. The enlarged part 221a of the flange 221 may have a circumferential length that is generally equal to or slightly smaller than a circumferential length of the bayonet connector 202. When the bayonet protrusion 202 moves into a longitudinal portion of the counter bayonet connector 11, the enlarged part 221a of the flange 221 can also move into the longitudinal portion of the counter bayonet connector 11. However, when the bayonet protrusion 202 moves into a circumferential portion of the counter bayonet connector 11, the enlarged part 221a of the flange 221 can only stay in the longitudinal portion of the counter bayonet connector 11. Therefore, when the bayonet protrusion 202 rotates along the circumferential portion of the counter bayonet connector 11, the body 20 and the delivery member guard 21; 21′ are rotated relative to the lock member 22. The rotation for connecting the cassette unit 2 to the drive unit 1, therefore, causes the lock member 22 to rotate relative to the delivery member guard 21; 21′ from the locked position to the unlocked position.

It should be noted that, alternatively, for the purpose of attaching the lock member to the body, the body 20 can comprise a circumferential slot of the body 20 instead of the assembly track as mentioned above. The lock member can therefore be snap-fitted to the circumferential slot of the body 20. It should be noted that if the assembling method of attaching the lock member to the body is changed, the above-mentioned components related to the assembling can be also changed dependent on the selected assembling method. Furthermore, the transverse part 221b of the flange 221 of the lock member 22 is mainly designed for the assembling way, thus, the flange 221 can be modified to only have the enlarged part 221a.

In one example, the body 220 of the lock member 22; 22′; 22″ is a tubular body 220. The tubular body 220 of the lock member 22; 22′; 22″ encloses a portion of the delivery member guard 21; 21′. The tubular body 220 may be a generally cylindrical shaped element or a generally hemicylindrical shaped element, for example. In one example, the lock member comprises a ledge 222; 222′ extending in the circumferential direction relative to the longitudinal axis along a portion of an inner surface of the tubular body 220 of the lock member 22, as shown in FIGS. 4A-4B and FIGS. 6C-6E, and the distally directed surface of the lock member is a part of the ledge 222; 222′.

The ledge 222 can extend only in the circumferential direction relative to the longitudinal axis along a portion of the inner surface of the tubular body 220 of the lock member 22, as shown in FIG. 4A. In this example, the unlocked position is defined by a location of a rupture portion 223 of the ledge 222 on the inner surface of the tubular body 220 of the lock member 22 relative to the rest of the ledge 222, as shown in FIG. 4A. The rupture portion 223 means a portion where the ledge 222 stops continuously extending in the circumferential direction relative to the longitudinal axis L. Therefore, once the protrusion 214 is aligned with the rupture portion in the direction of the longitudinal axis L, the protrusion 214 can freely move in the direction of the longitudinal axis without being blocked by the ledge 222. The protrusion 214 of the delivery member guard 21; 21′ is configured to move along the ledge 222 of the lock member 22, when the lock member 22 moves from the locked position to the unlocked position. Once the protrusion 214 disengages from the ledge 222 or meets the ruptured portion 223 of the ledge 222, the delivery member guard 21; 21′ is free to move in the proximal direction of the body 20 of the sub-assembly. Alternatively, the ledge 222′ can extend in both the circumferential direction relative to the longitudinal axis and the direction of the longitudinal axis. In this example, the ledge 222′ can be helical-shaped, as shown in FIGS. 6C-6E. The helical-shaped ledge 222′ extends helically towards the proximal direction of the cassette unit 2 from a distal end to a proximal end. In this example, the protrusion 214; 214′; 214″ of the delivery member guard 21; 21′; 21″ is configured to move along helical-shaped ledge 222′ of the lock member 22 when the lock member 22 moves from the locked position to the unlocked position. Once the protrusion 214; 214′ moves from the distal end of the helical-shaped ledge 222′ to the proximal end of the helical-shaped ledge 222′ of the helical-shaped ledge 222′, the delivery member guard 21; 21′ is free to move in the proximal direction of the body 20 of the sub-assembly. The exact position that defines unlocked position of the lock member 22 is dependent on the design of the medicament delivery device comprising the sub-assembly of the invention. In this example, even if the end-user rotates the lock member 22 to the unlocked position, the end-user still can change the mind and rotate the lock member back to the locked position. It should be noted that, the protrusion 214′ may comprise a proximally directed ramp surface.

Alternatively, the lock member 22 comprises a recess extending in the circumferential direction relative to the longitudinal axis L along a portion of the inner surface of the tubular body 220 of the lock member 22. The recess radially faces toward the longitudinal axis L. The distally directed surface of the lock member is a part of the recess so that the protrusion 214 of the delivery member guard 21; 21′ is configured to protrude into the recess. In this example, the protrusion of the delivery member guard 21; 21′ will be blocked from moving in either the distal direction or the proximal direction, relative to the body 20 of the sub-assembly, when the lock member 22 is in the locked position. Similarly, the recess comprises a rupture portion similar as above example to define the unlocked position of the lock member 22.

FIG. 6A illustrates the lock member 22 in the locked position. When the lock member 22 in the locked position, the protrusion 214 of the delivery member guard 21; 21′ rests on/moves along the ledge 222 of the lock member, so that the delivery member guard 21; 21′ is locked in the distal position; namely, the delivery member guard 21; 21′ is prevented from moving towards the proximal end of the body 20 relative to the lock member 22. FIG. 6B illustrates the lock member 22 in the unlocked position. In this example, the lock member 22 is rotated around the longitudinal axis L relative to the delivery member guard 21; 21′. In examples where the ledge is a partially circular ledge 222, as shown in FIG. 4A, since the lock member 22 only partially extends along the inner surface of the tubular body 220 of the lock member 22. Once the protrusion 214 of the delivery member guard 21; 21′ is no longer aligned with a portion of the ledge 222 in the direction of the longitudinal axis L, the distally directed surface of the lock member 22 is spaced apart from the proximally directed surface of the delivery member guard 21; 21′. The position that the protrusion 214 of the delivery member guard 21; 21′ is fully misaligned with a portion of the ledge 222 in the longitudinal axis L therefore defines the unlocked position of the lock member 22. The exact unlocked position of the lock member 22 can therefore be defined by a choice of a length of the ledge 222, dependent on design requirements.

The movement of the delivery member guard 21; 21′ from the distal position to the proximal position can be used as an “unlock” mechanism of the cassette unit 2, so that an injury or contamination of the medicament delivery member caused by being e.g. unintentionally touched by a user or falling to the ground can be prevented. For example, the delivery member guard 21; 21′ may be able to move into a second distal position when the delivery member guard 21; 21′ has already moved into the proximal position from the distal position; further detail regarding this initiation mechanism will be explained later. Alternatively, the movement of the delivery member guard 21; 21′ from the distal position to the proximal position can be used in a cassette unit that the end-user needs to self-attach to a medicament delivery member. For example, the end-user may need to self-attach a pen needle to the cassette unit. In this example, the delivery member guard should be in the distal position at the beginning, so that the end-user is able to attach the pen needle onto the cassette unit; when the end-user attaches the pen needle onto the cassette unit, the end-user can move the lock member 22 from the locked position to the unlocked position, so that the delivery member guard 21; 21′ can move from the distal position to the proximal position, e.g. by simply being pulled by the user or pushed by a biasing member or resilient structure, so that the attached pen needle can be covered by the delivery member guard 21; 21′. Alternatively, if the sub-assembly is used in a cassette unit with a preassembled medicament delivery member set, the proximal movement of the delivery member guard 21; 21′ from the distal position of the delivery member guard 21; 21′ can be used to enable the user to manipulate the medicament delivery member set for mounting the medicament delivery member; this alternative will be explained in detail later.

In preferred example, a safety mechanism can be provided to prevent the end-user from accidentally detaching cassette unit 2. The safety mechanism is provided by the tubular body 220 of the lock member 22 comprises a cut-out/recess 220a′ opening in the direction transverse to the longitudinal axis L, and a locking tab 20a′ extending from a wall of the body 20′, as shown in FIGS. 33-36D. The cut-out/recess 220a′ is configured to radially face towards the distal end 20b′ of the locking tab 20a′.

In this example, the body 20′ comprises the locking tab 20a′ distally extending from the wall of the body 20′ to a distal end 20b′ of the locking tab 20a′. The locking tab 20a′ is flexible. As shown in FIGS. 36A-36D, when the end-user attaches the cassette unit 2 to the drive unit 1 (the operation sequence shown with arrow S) via the bayonet connection as mentioned above, the body 20′ rotates (as shown with arrow R3 in FIGS. 36B-36C) relative to the lock member 22′. The distal end 20b′ of the locking tab 20a is deflected radially inward relative towards the longitudinal axis L by the tubular body 220 of the lock member 22′ (as shown with arrow Z in FIG. 36C) when the body 20′ rotates relative to the lock member 22′, as shown in FIGS. 36B-C. When the cassette unit 2 is properly attached to the drive unit 1, the distal end 20b′ of the locking tab 20a′ radially algins with the cut-out/recess 220a′ of the lock member 22′, thus, the locking tab 2 flexes radially outward relative to the longitudinal axis L, and positions into the cut-out/recess 220a′, as shown in FIG. 36D. When the locking tab 2 is in the cut-out/recess 220a′, the lock member 22′ and the body 20′ are prevented from rotating relative to each other.

As mentioned above, the enlarged part 221a of the lock member 22′ will stay in the longitudinal portion of the counter bayonet connector 11 of the drive unit 1 when the bayonet protrusion of the body is in the circumferential portion of the counter bayonet connection of the drive unit 1, meaning that the cassette unit 2 is properly attached to the drive unit 1. Therefore, when the lock member 22′ and the body 20′ are preventing from rotating relative to each other as mentioned above, the cassette unit 2 cannot be detached from the drive unit 1 via the bayonet connection.

In a preferred example, the locking tab 20a′ comprises a protrusion 20c′ extending from an outer surface of the locking tab 20a′ in the direction transverse to the longitudinal axis L. In this example, when the end-user wants to detach the cassette unit 2 from the drive unit 1, the end-user can press on the protrusion 20c′ radially inward to the longitudinal axis L, so that the locking tab 20a′ deflects radially inward to the longitudinal axis L. Thereby, the distal end 20b′ moves out from the cut-out/recess 220a′ of the lock member 22′. As the distal end 20b′ moves out from the cut-out/recess 220a′ of the lock member 22′, the lock member 22′ and the body 20′ can rotate relative to each other, so that the cassette unit 2 can be detached from the drive unit 1 by rotating the cassette unit 2 along the bayonet connection.

In a preferred example, the sub-assembly comprises a distal lid 24; 24′ as a part of the body 20 of the sub-assembly, as shown in FIG. 6G and FIGS. 7-8. The distal lid 24; 24′ is configured to fixedly attach to the distal end of the body 20, for example by a snap-fit between a snap aperture 204 on the body 20 and a snap arm 241; 241′ on the distal lid 24; 24′, so that a movement of the medicament container in the distal direction relative to the body 20 can be blocked. The distal lid 24 can also be fully or at least partially received within the longitudinal part 203a of the assembly track 203 so that the lock member 22 can only be rotatable relative to the body 20 along the transverse part 203b of the assembly track 203. In this example, the distal lid 24 comprises a proximally extending arm 242 configured to fit into the longitudinal part 203a of the assembly track 203.

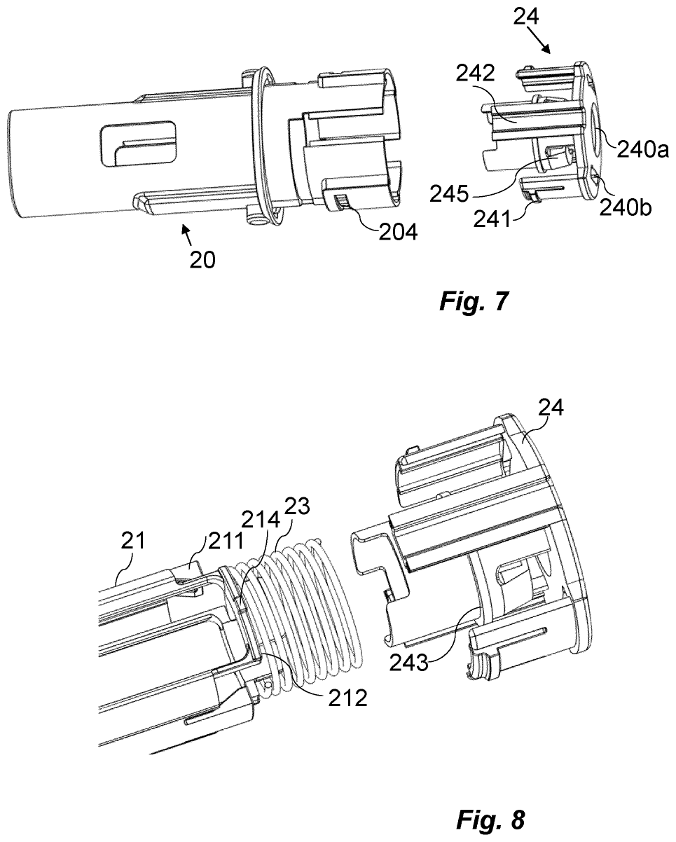

The shape of the distal lid 24; 24′ is typically dependent on the shape of the distal end of the body 20. The distal lid 24; 24′ comprises a lid body 240; 240′, as shown in FIG. 2, FIG. 6G and FIG. 7; the lid body 240; 240′ can be formed in accordance with the shaped of the medicament container received within the body 20, for example, the lid body 240; 240′ comprises a tubular portion. Further, the distal lid 24; 24′ typically comprises an opening 240a axially aligned with a portion of the distal end of the medicament container, so that when the cassette unit 2 is attached to the drive unit 1, the plunger rod is able to pass through the opening 240a and act on the medicament container for delivering the contained medicament.

In one example, the distal lid 24 may comprise a curve-shaped opening/slot 240b (when observed from the axial direction), so that the arm of the delivery member guard 21 or an interaction part 251 of an actuator 25 (will be explained later) can pass through the curve-shaped opening/slot 240b and protrude into the drive unit 1. In another example, the distal lid 24 may be a generally rectangular or oval-shaped shape. In this example, the arm of the delivery member guard 21 or the interaction part 251 of the actuator 25 can pass through from a space between the distal edge of the body 20 and an edge of the distal lid 24.

It should be noted that the cassette unit sub-assembly can be used with a medicament delivery device that is triggered by a button, e.g. arranged at a distal end of the drive unit 1 or a side body wall of the drive unit 1. In this example, the neither the delivery member guard nor the actuator involved in triggering the medicament delivery operation. Therefore, the distal lid does not need to comprise the curve-shaped opening/slot.

The distal lid 24; 24′ optionally comprises a label, e.g. RFID tag, bar code, QR code, or mechanical code, for containing information about the cassette unit 2 or the contained medicament, so that a reader in the drive unit 1 may get the information for further use.

The distal lid 24 optionally comprises a container support 245 for supporting the medicament container received within the body 20. The container support 245 comprises a proximally directed surface. In one example, the container support 245 is a flexible arm 245. The flexible arm 245 can be formed by a cut-out, opening towards the direction transverse to the longitudinal axis L, on the lid body 240; alternatively, the flexible arm 245 may extend from the lid body 240 towards the distal direction. In this example the container support 245 can prevent an axial movement of the medicament container received within the body 2 in the distal direction. The proximally directed surface of the container support 245 can be formed on an inner surface of the flexible arm; or can be formed on a distal tip of the flexible arm. The container support 245 may also be used to tolerate the received medicament container with different dimension, e.g. certain length and/or width of the received medicament container, regarding to the engineering tolerance.

The sub-assembly of the invention optionally comprises a biasing member 23 arranged between a first distally directed surface of the delivery member guard 21; 21′ and a proximally directed surface of the body 20. The biasing member 23 can be a spring or a flexible arm. The biasing member 23 is configured to bias the delivery member guard 21; 21′ towards the proximal end of the body 20. In one example, the first distally directed surface of the delivery member guard 21; 21′ is defined by a part of the ledge 212; 212′. In examples where the sub-assembly comprises the distal lid 24; 24′, the proximally directed surface 243 of the body 20 can be arranged on the distal lid 24; 24′, as shown in FIG. 8. Alternatively, the body may be arranged with an inner ledge extending along an inner surface of the body, so that the proximally directed surface can be arranged on the inner ledge.

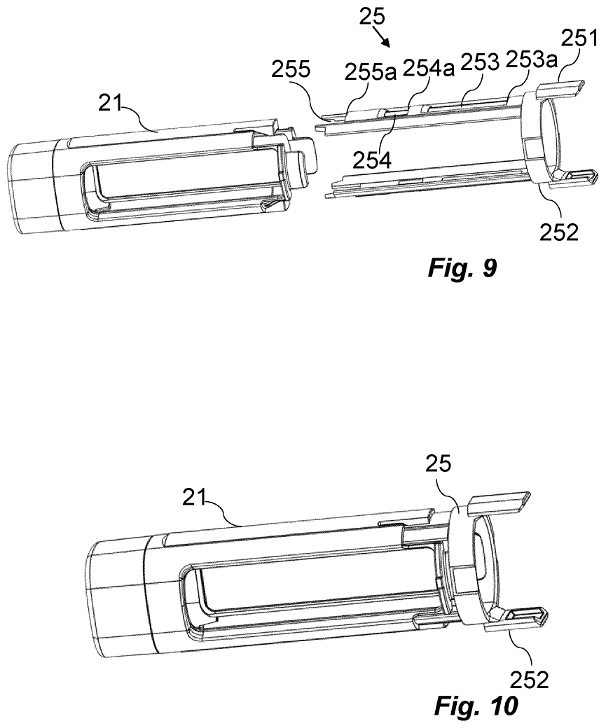

The sub-assembly of the invention optionally comprises the actuator 25 movable relative to the body 20 along the longitudinal axis L between a proximal position and a distal position, as shown in FIG. 2. In a preferred example, the actuator 25 comprises an elongated body extending along the longitudinal axis between a proximal end and a distal end. The actuator 25 comprises an interaction part 251, a retaining part 252, a distal cut-out 253. In the first example, as shown in FIG. 9, the actuator 25 further comprises a second cut-out 254 and a third cut-out 255. In the second example, as shown in FIG. 16, instead of the second cut-out 254 and the third cut-out 255, the actuator 25 comprises a recess 255′ arranged on an outer surface of the elongated body of the actuator 25. The difference between the two examples of the actuator 25 will only be relevant to a lock-out mechanism for the delivery member guard 21, namely, a mechanism that prevents the delivery member guard 21 from moving in the distal direction relative to the body 20 after the cassette unit 2 has been used; this part will be explained in detail later.

The interaction part 251 of the actuator 25 is configured to interact with a component of the drive unit 1 of the medicament delivery device comprising the sub-assembly. For example, the drive unit 1 may comprise a motor, motor-driven gear, a plunger rod, and a mechanical switch for switching on the motor. The interaction part 251 of the actuator 25 may be in contact with the mechanical switch and move the switch to switch on the motor when the delivery member guard 21 moves in the distal direction relative to the body 20 and the drive unit 1. In this case, if the switch is designed to face toward the proximal end of the drive unit 1, the switch may be placed within a slot of the drive unit 1 (for preventing any unintentional contact) and the interaction part 251 may be a rod with a diameter that is able to insert into such hole and press on the switch. Alternatively, the switch may be arranged on an inner surface of the drive unit 1. In this case, the interaction part 251 may be a radially outwardly protruding arm, so that the interaction part 251 can press on the switch or flip the switch over when the interaction part is aligned with the switch or moved past the switch. In this example, the arm 211 of the delivery member guard 21 may not protrude into the drive unit 1 of the medicament delivery device comprising the sub-assembly. In this example, the delivery member guard 21 comprises a second distally directed surface which is preferably arranged on a distally directed surface of the arm 211. The second distally directed surface is configured to interact with the actuator 25. As shown in FIG. 10, the actuator 25 is movably attached to the delivery member guard 21.

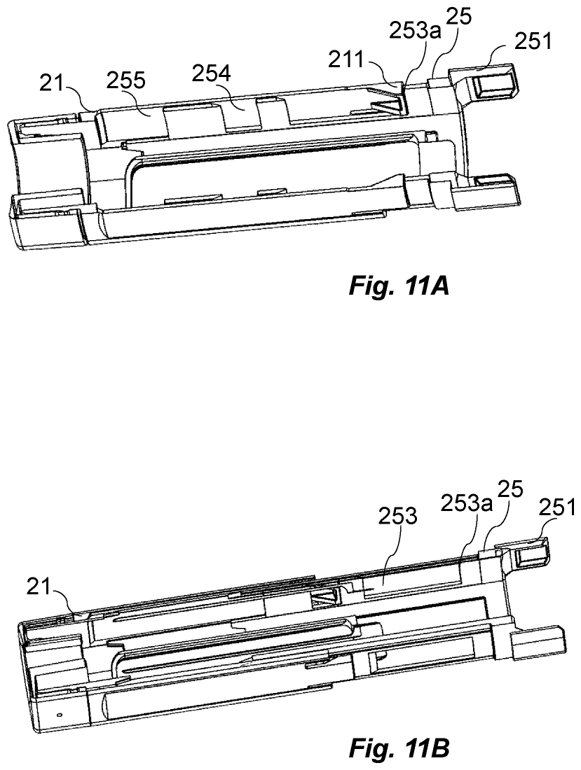

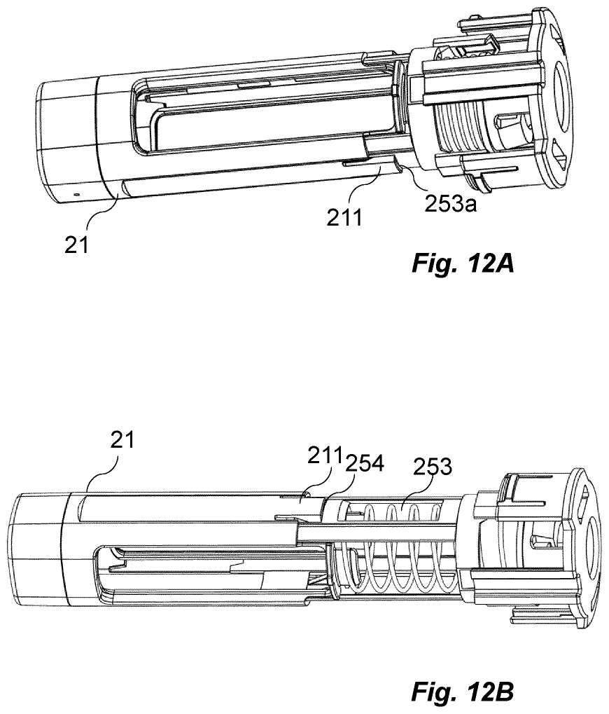

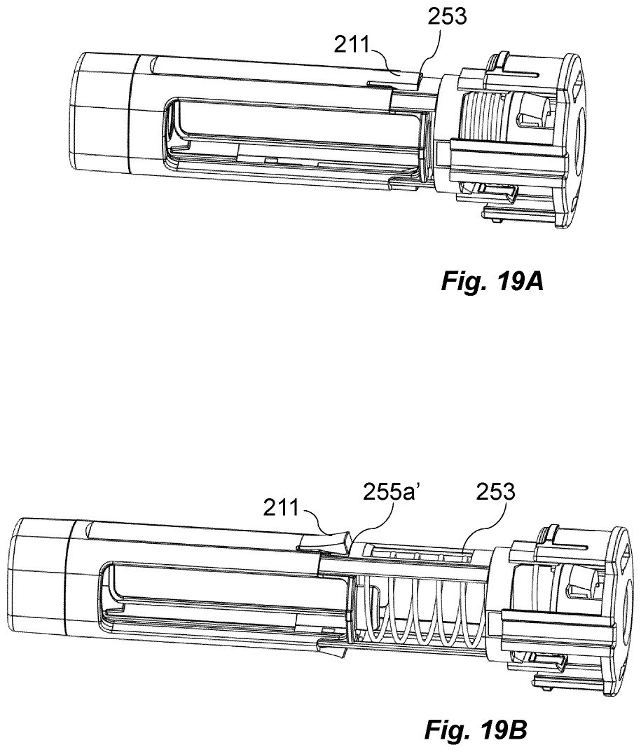

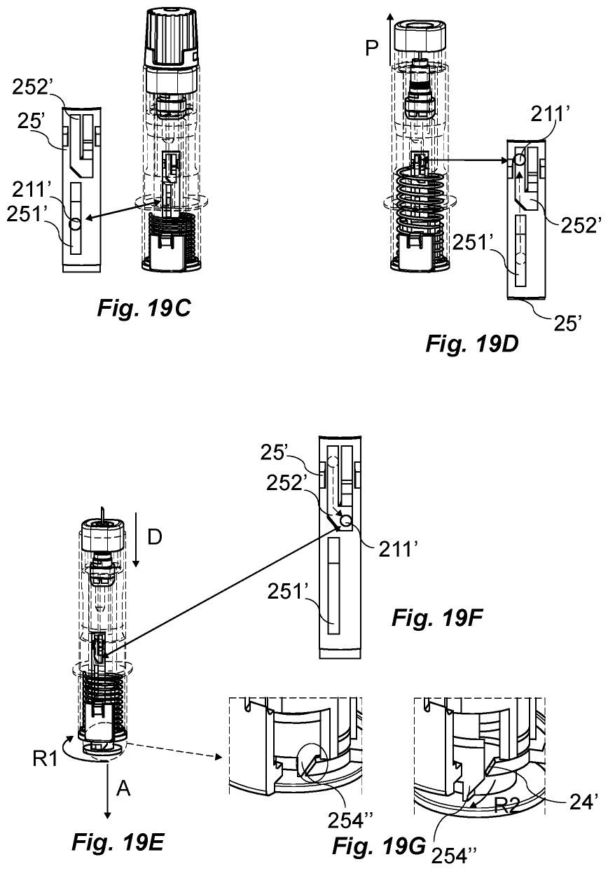

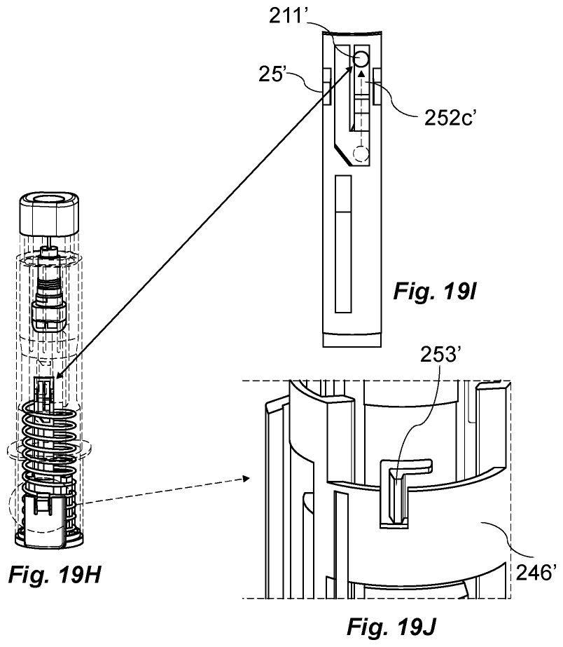

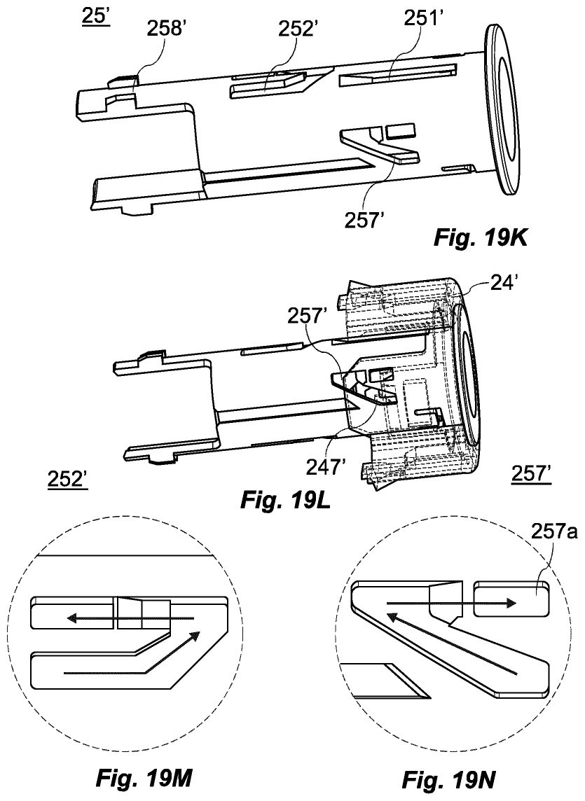

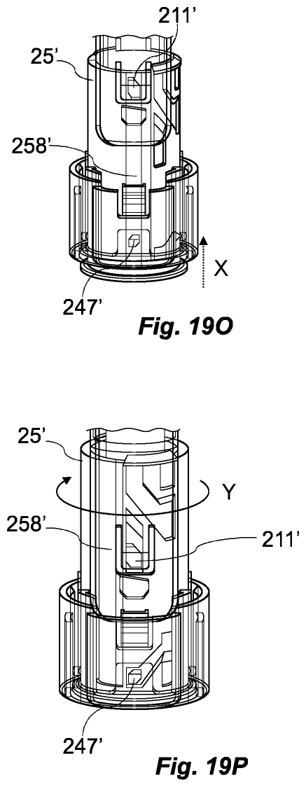

The actuator 25 is positioned initially in its proximal position. As shown in FIG. 11A and FIG. 12A and FIG. 19A, the actuator 25 is in its proximal position when the delivery member guard 21 is locked in the distal position. The arm 211 of the delivery member guard 21 is adjacent to a distal end edge 253a of the first cut-out 253 of the actuator 25. The delivery member guard 21 is released and moved to the proximal position when the lock member 22 is in the unlocked position and the delivery member guard is subjected to a proximally biasing force. When the delivery member guard 21 is in the proximal position, as shown in FIG. 11B and FIG. 12B/FIG. 19B, the actuator 25 is in its proximal position. The arm 211 of the delivery member guard 21 now moves proximally into the second cut-out 254 of the actuator 25. The arm 211 of the delivery member guard 21 may be flexible in the radial direction relative to the longitudinal axis L, so that the arm 211 can flex out to move past the ledge between the first cut-out 253 of the actuator 25 and the second cut-out 254 of the actuator 25. Alternatively, the ledge between the first cut-out 253 of the actuator 25 and the second cut-out 254 of the actuator 25 can be flexible in the radial direction relative to the longitudinal axis L, so that when the delivery member guard 21 moves in the proximal direction relative to the body 20, the arm 211 can deflect the ledge and move past the ledge of the actuator 25 from the first cut-out 253 of the actuator 25 to the second cut-out 254 of the actuator 25.

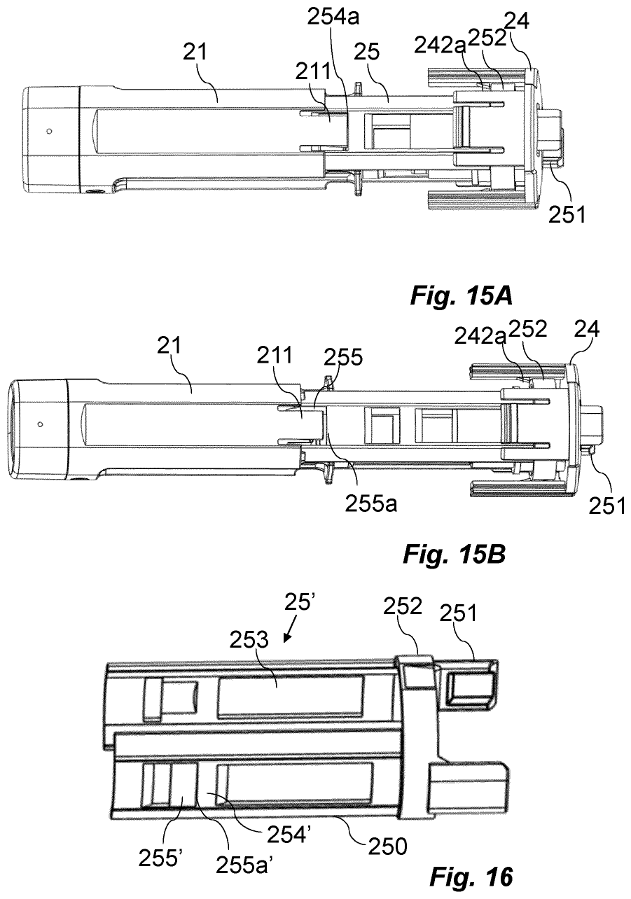

When the arm 211 of the delivery member guard 21 is in the second cut-out 254 of the actuator 25, the arm of the delivery member guard 21 is adjacent to a distal end edge 254a of the second cut-out 254 so that a further distal movement of the delivery member guard 21 can move the actuator 25 from the proximal position of the actuator 25 to the distal position of the actuator 25, as shown in FIG. 15A. Therefore, when the actuator 25 moves from its proximal position to its distal position, the interaction part 251 of the actuator 25 can move into the drive unit 1 of the medicament delivery device and interact with a part within the drive unit 1.

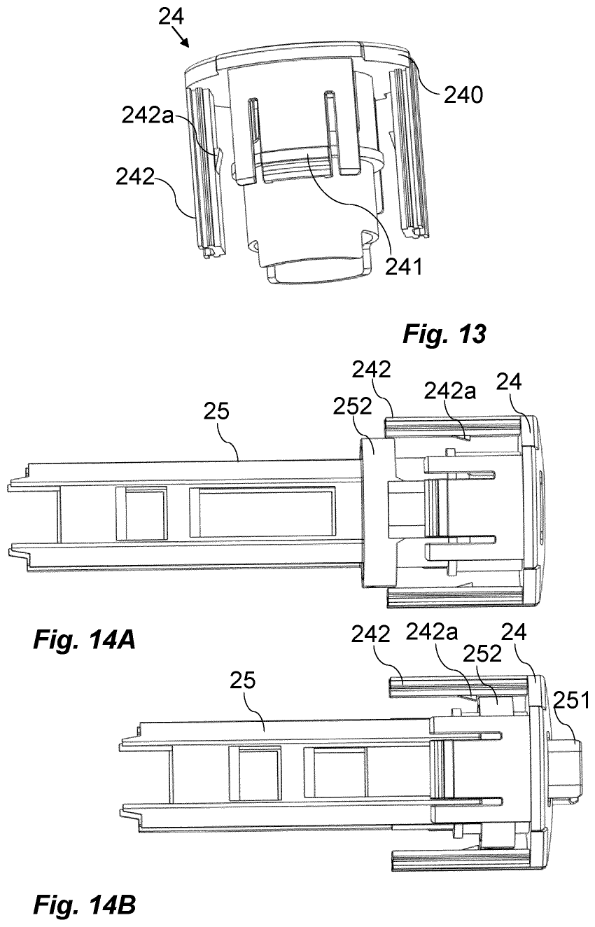

In another example, the actuator 25 is configured to be retained in its distal position. In this example, the body 20 comprises a retaining portion providing a distally directed surface adjacent to a proximally directed retaining surface of the actuator 25 once the proximally directed retaining surface of the actuator 25 moves past the retaining portion of the body. In the example, as shown in FIG. 13 and FIGS. 14A-14B, the retaining portion 242a of the body 20 can be arranged on the inner surface of the proximally directed arm 242 of the distal lid 24. The proximally directed retaining surface of the actuator 25 is defined by the retaining part 252 of the actuator 25. The design as described in this example can be used with a medicament delivery device with a drive unit using the distal movement of the interaction part 251 of the actuator 25 to switch on a set of control electronics, so that once the end-user moves the delivery member guard 21 and the actuator 25 in the distal direction relative to the body 20, the control electronics will be switched on and will only be switched off once the cassette unit 2 comprising the sub-assembly has been removed from the drive unit 1. In this example, the actuator 25 will be retained in its distal position even if the delivery member guard 21 is moved in the proximal direction relative to the body 20 again, as shown in FIG. 15B. Furthermore, when the actuator 25 is retained in its distal position, the interaction part 251 of the actuator 25 will therefore be protruding from the distal lid 24, so that a visual and/or tactile indication can be provided to the end-user for indicating that the cassette unit 2 has been used when the end-user detaches the cassette unit 2 from the drive unit 1.

For initiating an operation of the medicament delivery device with the drive unit 1 and the cassette unit 2, the delivery member guard 21 is moved by the end-user towards the drive unit 1 to a distal trigger position. This distal movement of the delivery member guard 21 reveals a medicament delivery member that connects with a contained medicament and may also trigger the drive unit 1 to output a force on the contained medicament. Therefore, when the delivery member guard 21 is in the distal trigger position, the medicament delivery member is revealed to the user. This distal movement of the delivery member guard 21 can be performed by pressing a proximal end of the front shield 210 of the delivery member guard 21 on a medicament delivery site.

In examples where the sub-assembly comprises the biasing member 23, the biasing member 23 is configured to bias the delivery member guard 21 in the proximal direction relative to the body 20 of the sub-assembly. Once the medicament delivery operation has been performed, the end-user may remove the medicament delivery device from the medicament delivery site, and the biasing member 23 can therefore bias the delivery member guard 21 in the proximal direction of the body 20 so that the front shield 210 of the delivery member guard 21 can shield the medicament delivery member.

The delivery member guard 21 is therefore biased to a proximal after-use position by the biasing member 23. The proximal after-use position can be the proximal position of the delivery member guard 21 as mentioned above or can be a second proximal position than above mentioned the proximal position of the delivery member guard 21, dependent on the design of the medicament delivery device. For example, if the medicament delivery member of the medicament delivery device is configured to move beyond its initial position in the proximal direction of the medicament delivery device after use, like in a medicament delivery device comprising an auto needle insertion mechanism; the delivery member guard may also move into the second proximal position as a proximal after-use position.

In the example, as shown in FIG. 15B, when the delivery member guard 21 moves into the proximal after-use position, the actuator 25 is retained in its distal position, the arm 211 of the delivery member guard 21 is therefore move into the third cut-out 255 of the actuator 25. The actuator comprises a proximally directed lock-out surface 255a which is preferably arranged on the distal end edge 255a of the third cut-out 255, so that when the delivery member guard 21 is in the proximal after-use position, the second distally directed surface of the delivery member guard 21 is adjacent to the proximally directed lock-out surface 255a of the actuator 25. Therefore, any further movement of the delivery member guard 21 in the distal direction relative to the body 20 is prevented.

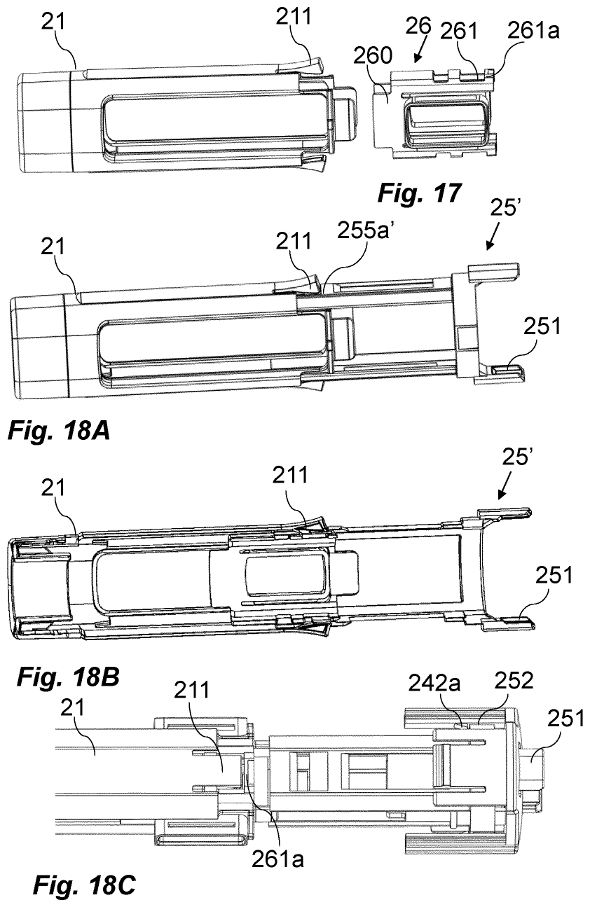

In the second example of the actuator 25, as shown in FIG. 16, the sub-assembly comprises an inner housing 26. As shown in FIG. 17, the inner housing 26 is radially arranged relative to the longitudinal axis between the delivery member guard 21 and the actuator 25. The inner housing 26 is fixed to the body 20. The inner housing 26 comprises a body 260 with a distal cut-out 261. In this example, the recess 255′ of the actuator 25 is configured to block the arm 211 of the delivery member guard 21 from moving into the cut-out 261 of the inner housing when the actuator 25 is in its proximal position, as shown in FIGS. 18A-B. The arm 211 of the delivery member guard 21 is adjacent to a distal end edge of the recess 255a′ of the actuator 25 when the delivery member guard 21 is in the proximal position. When the delivery member guard 21 moves the actuator 25 to the distal position of the actuator 25, the actuator 25 is retained in its distal position by the body 20, so that once the delivery member guard 21 moves to the proximal after-use position, the arm 211 of the delivery member guard 21 can move into the cut-out 261 of the inner housing 26, as shown in FIG. 18C. In this example, the second distally directed surface of the delivery member guard 21 is adjacent to a proximally directed surface on a distal end edge 261a of the cut-out 261 of the inner housing 26. Therefore, any further movement of the delivery member guard 21 in the distal direction relative to the body 20 is prevented.

In another example, the actuator 25′ comprises an actuator body 250′, and a track. The track comprises a first track 251′ and a second track 252′. The first and the second track 251′, 252′ are arranged on a wall of the actuator body 250′. The first and the second track 251′. 252′ are configured to interact with a protrusion arranged on the delivery member guard. In a preferred example, the delivery member guard 21′ comprises a protrusion 211′, preferably, extending from a wall of the delivery member guard 21′ in the direction transverse to the longitudinal axis L, as shown in FIG. 6C. In this example, the actuator 25′ is rotatable relative to the delivery member guard 21′ around the longitudinal axis L. As shown in FIG. 19C, the protrusion 211′ of the delivery member guard 21′ is initially positioned within the first track 251′ of the actuator 25′, when the lock member 22 is in the locked position. When the lock member 22 is in the unlocked position, the protrusion 211′ can move together with the delivery member guard 21′ in the proximal direction (arrow P) and therefore enters into the second track 252′ of the actuator 25′ when the delivery member guard 21′ is in its proximal position (the dash-line arrow), as shown in FIG. 19D. The proximal end of the first track 251′ optionally comprises a ramp surface facing towards the distal end of the actuator 25′. Alternatively, the proximal end of the protrusion 211′ of the delivery member guard 21′ optionally comprises a ramp surface facing towards the proximal end of the actuator 25′. When the delivery member guard 21′ is moved in the distal direction (arrow D) relative to the body 20, the protrusion 211′ of the delivery member guard 21′ will move along a longitudinal portion 252a′ of the second track 252′ towards a chamfer surface 252b′, so that the actuator 25′ can be rotated (arrow R1) and axially moved (arrow A) in the distal direction relative to the body 20 by the delivery member guard 21′, as shown in FIGS. 19E-F. The actuator 25′ can be arranged with one or more interaction parts for interacting with the drive unit 1, similar as the interaction part 251 mentioned above.

Optionally, the actuator 25′ comprises an inclined surface 254′ arranged at the distal end of the actuator 25′, as shown in FIG. 6F. The inclined surface 254″ is angled related to the longitudinal axis L, and is configured to interact with a counter inclined surface on the distal lid 24′, as shown in FIG. 19G, such interaction between the inclined surface 254″ of the actuator 25′ and the counter inclined surface can thus facilitate the rotation (arrow R2) between the actuator 25′ and the delivery member guard 21′ (the actuator 25′ can be easily rotated by the delivery member guard 21′). The second track 252′ optionally comprises a lockout portion 252c′ for providing a delivery member guard lock-out mechanism. In this example, as shown in FIG. 191, in this example, once the delivery member guard 21′ moves to the proximal after-use position, the protrusion 211′ of the delivery member guard 21′ will move into the lockout portion 252c′ together with the distal movement of the delivery member guard 21′. In this example, the protrusion 211′ should be arranged at a flexible portion of the delivery member guard 21′, e.g. flexible arm or a part formed by a flexible material; or a distal frame of the lockout portion 252c′ of the actuator 25′ should be flexible. Furthermore, the actuator 25′ optionally comprises a locking tab 253′ configured to move into a locking cut-out 246′ of the distal lid 24′ when the delivery member guard 21′ moves to the proximal after-use position, as shown in FIGS. 6F-6G and FIG. 19J. It should be noted that, at least one of above-mentioned the arrangements for the delivery member guard lock-out mechanism is enough to perform a delivery member guard lock-out mechanism when the delivery member guard 21; 21′ moves to the proximal after-use position. However, multiple above-mentioned the arrangements for the delivery member guard lock-out mechanism can also be used together. For example, the second track 252′ of the actuator 25′ can have a lockout portion 252c′ interacting with the protrusion 211′ of the delivery member guard 21′ and have the locking tab 253′ interacting with the locking cut-out 246′ of the distal lid 24′.

In another example, as shown in FIGS. 19K-19L, instead of the inclined surface 254″ for interacting with the locking cut-out, the actuator 25′ comprises a distal track 256′ and the distal lid 24′ comprises a protrusion 247′ positioned within the distal track. When the delivery member guard 21; 21′ moves in the distal direction relative to the actuator 25′, the actuator 25′ rotates, as mentioned above. The distal track 256′ also facilitates the rotator of the actuator 25′ by the interaction between the distal track 256′ and the protrusion 247′ of the distal lid 24′, as shown in FIGS. 19M-19N.

FIG. 19M shows the second track 252′, when the delivery member guard 21; 21′ moves in the distal direction relative to the body, and then moves back in the proximal direction relative to the body, the moving path of the protrusion 211′ of the delivery member guard 21; 21′ moves, as shown with the arrow in FIG. 19M, along the second track. In the meanwhile, as shown in FIG. 19N, the protrusion 247′ of the distal lid 24′ moves relative to the actuator 25′ along the distal track 257′ (the moving path as shown with the arrow in FIG. 19N). In another example, instead of the locking tab 253′ of the actuator 25′ and the locking cut-out 246′ of the distal lid 24′ as mentioned above, the actuator 25′ comprises a distal cut-out/recess 257a′ for holding the actuator 25′. In this example, once a distal cut-out/recess 257a′ of the actuator 25′ aligned with the protrusion 247′ of the distal lid 24′, namely, the protrusion 247′ is positioned within the distal cut-out/recess 257a′, the distal lid 24′ and the actuator 25′ fix to each other in the direction of the longitudinal axis L. It should be noted that the protrusion can be placed on the body rather than the distal lid.

It should be noted that, the first track and the second track can be formed by cut-outs or recess on the body of the actuator; or can be formed by multiple ledges extending from the body of the actuator in the direction transverse to the longitudinal axis L. Furthermore, the protrusion of the delivery member guard can be replaced by a distally directed arm arranged on the delivery member guard. For example, the distally directed arm extends from the front shield of the delivery member guard in the distal direction of the housing, and a distal end of the distally directed arm is configured to act on the chamber of the second track of the actuator, so that the actuator can be rotated when the delivery member guard moves in the distal direction relative to the actuator along the longitudinal axis L. In this example, the first track of the track of the actuator and the lockout portion of the second track are not necessary for the sub-assembly of the invention.

It should be noted that the delivery member guard lock-out mechanism may be crucial in some cases from a medicament delivery device regulation aspect, but is not from a mechanical perspective—the disclosed delivery member guard lock-out mechanism is only an optional feature to the applied invention. There are many known variations of the delivery member guard lock-out mechanism that can be implemented to the sub-assembly of the invention without any substantial modification of the sub-assembly.

Furthermore, in a preferred example, a slot can be arranged on the actuator 25′ for assembly purpose. As shown in FIGS. 190-19P, the slot 258′ on activator 25′. The protrusion 211′ of medicament delivery device 21′ and the protrusion 247′ of distal lid 24′ are both inserted into the slot 258′, move a linear distance to an assembling position (shown with arrow X in FIG. 19O, the distance is dependent on the dimension of the components). Once the protrusion 211′ of medicament delivery device 21′ and the protrusion 247′ of distal lid 24′ in the assembling position, the actuator 25′ can be twisted (arrow Y in FIG. 19P), then the protrusion 211′ of medicament delivery device 21′ and the protrusion 247′ of distal lid 24′ are situated into the first and the second track 251′, 252′.

It should be noted that, as the actuator 25; 25′ is only related to the delivery member guard lock-out mechanism and/or a trigger mechanism by the distal movement of the delivery member guard. As mentioned above, the delivery member guard lock-out mechanism may be crucial in some cases from a medicament delivery device regulation aspect, but is not from a mechanical perspective; also, the cassette unit sub-assembly can be used with a medicament delivery device that is triggered by the button of the drive unit 1. Therefore, the actuator 25; 25′ is only an optional feature for the sub-assembly of the invention, namely, the initial locking mechanism of the delivery member guard that is achieved by the lock member and the delivery member guard.

The sub-assembly of the invention can be operated by the end-user with the steps of: attaching the cassette unit 2 to the drive unit 1, preferably through the bayonet connection between the cassette unit 2 and the drive unit; and releasing the delivery member guard 21; 21′; 21″ to move in a proximal direction relative the medicament delivery device by moving the lock member 22; 22′; 22″ relative to the delivery member guard 21; 21′; 21″ from the locked position to the unlocked position, preferably by rotating the lock member 22; 22′; 22″ relative to the delivery member guard 21; 21′; 21″ while the end-user rotates the cassette unit 2 to the drive unit 1 for connecting the cassette unit 2 to the drive unit 1 through the bayonet connection.

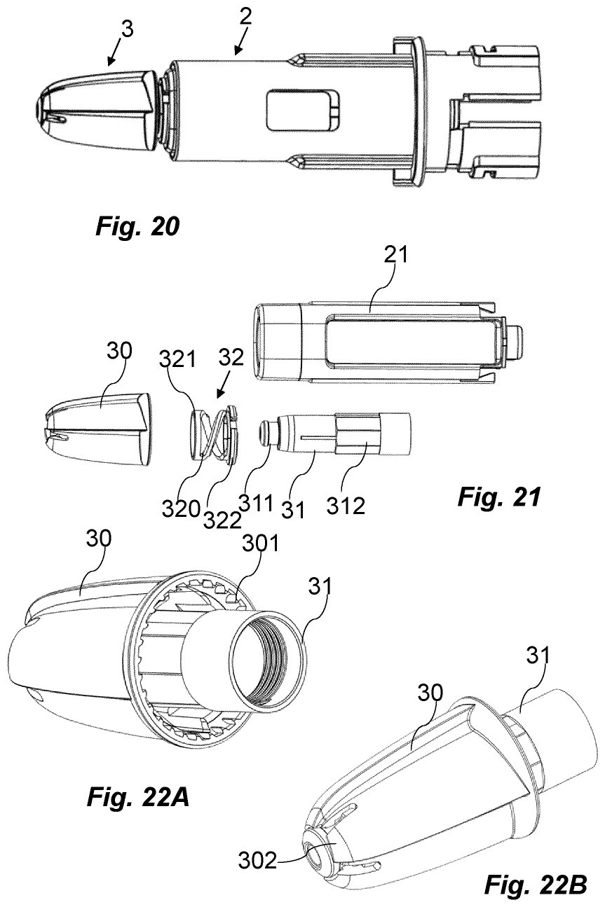

The sub-assembly of the invention can be used with a cassette unit 2 with a preassembled medicament delivery member set 3; 3′; 3″, as shown in FIG. 20, FIG. 26A and FIG. 27. The preassembled medicament delivery member set 3; 3′; 3″ is fixed to the proximal end of the body. For example, the preassembled medicament delivery member set 3; 3′; 3″ can be attached to the body. Alternatively, in the example that the cassette unit 2 comprises the inner housing, the preassembled medicament delivery member set 3; 3′; 3″ can be attached to the inner housing. Furthermore, in one example where the cassette unit 2 comprises a support unit for receiving and/or supporting a medicament container arranged within the body 20, the preassembled medicament delivery member set 3; 3′; 3″ can be attached to the support unit. In one example, the preassembled medicament delivery member set 3; 3′; 3″ comprises a tubular outer cap 30; 30′; 30″, an inner cap 31, and a clutch member 32; 32′, as shown in FIG. 21 and FIG. 28. The inner cap 31 is configured to be releasably attached to the cassette unit 2 and can be detached from the cassette unit 2 by a rotation relative to the body 20 of the cassette unit 2. Preferably, the inner cap 31 is configured to fully cover a medicament delivery member.

The tubular outer cap 30; 30′; 30″ at least partially surrounds the inner cap 31. The clutch member 32; 32′ is longitudinally positioned between a proximal end of the delivery member guard 21; 21′; 21″ and the medicament delivery member set 3. The clutch member 32; 32′ is radially positioned relative to the longitudinal axis L between the tubular outer cap 30; 30′; 30″ and the inner cap 31. The clutch member 32; 32′ comprises a clutch biasing member 320; 320′, the clutch member 320; 320′ being axially movable relative to the outer cap 30; 30′; 30″ between a relaxed position and a tensioned position. The tubular outer cap 30; 30′; 30″ is axially fixed to and rotatable relative to the inner cap 31. For example, the inner cap 31 comprises a proximal annular recess 311, and the tubular outer cap 30; 30′; 30″ comprises a proximal gripper 302 engaging with the proximal recess 311 of the inner cap 31, as shown in FIG. 22B. The clutch member 32; 32′ is rotationally fixed to the inner cap 31. For example, the inner cap 31 may comprise a grip portion 312, and an inner surface of the clutch member 32; 32′ may comprise a counter grip portion 322a with a shape corresponding to the grip portion 312 of the inner cap 31, as shown in FIG. 24.

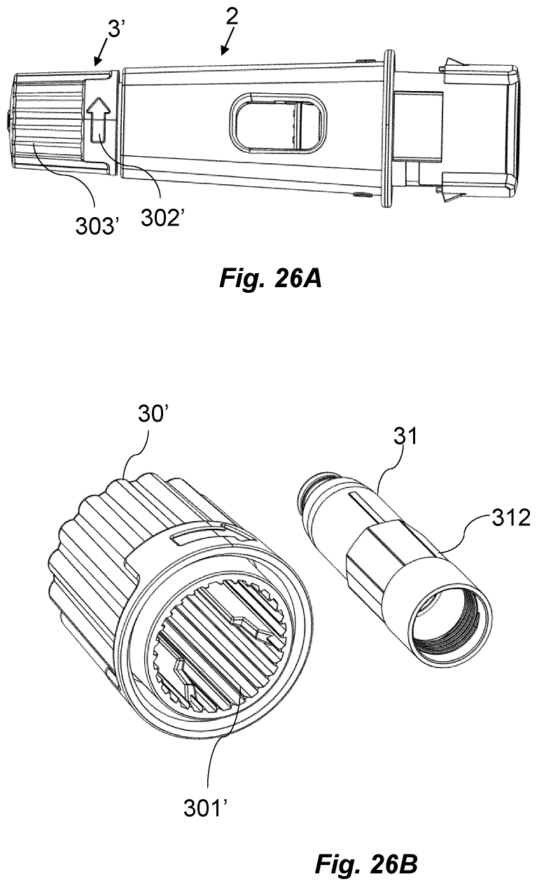

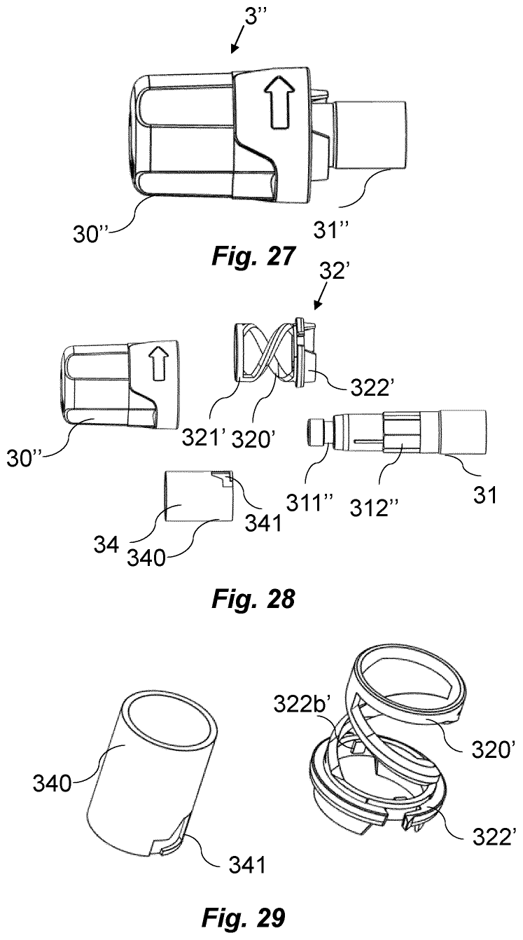

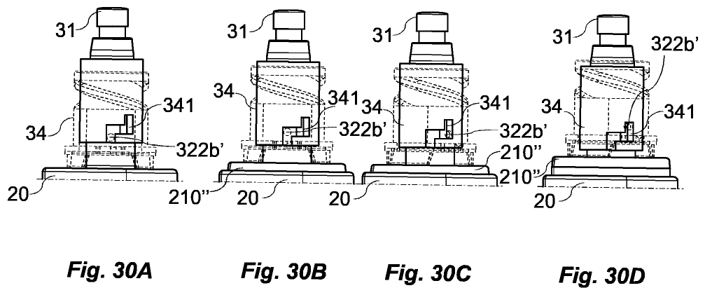

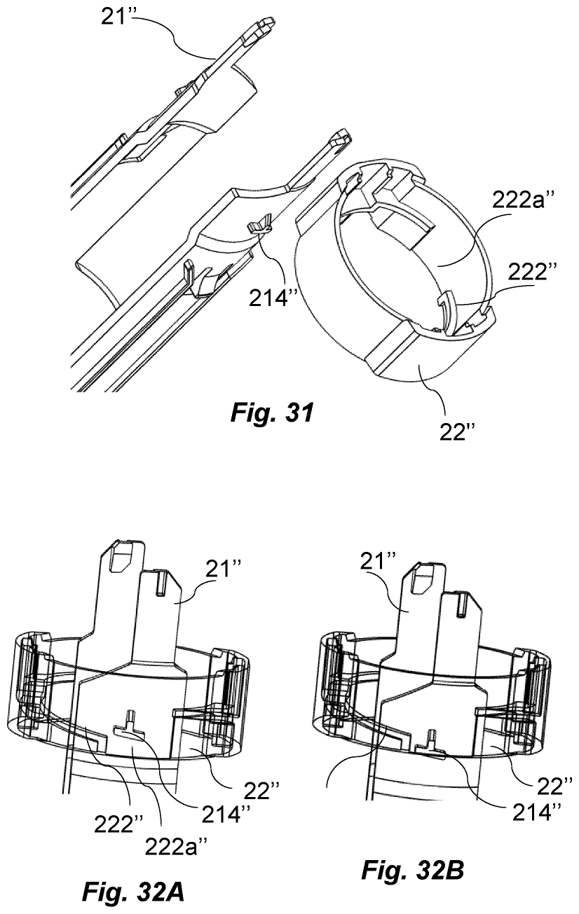

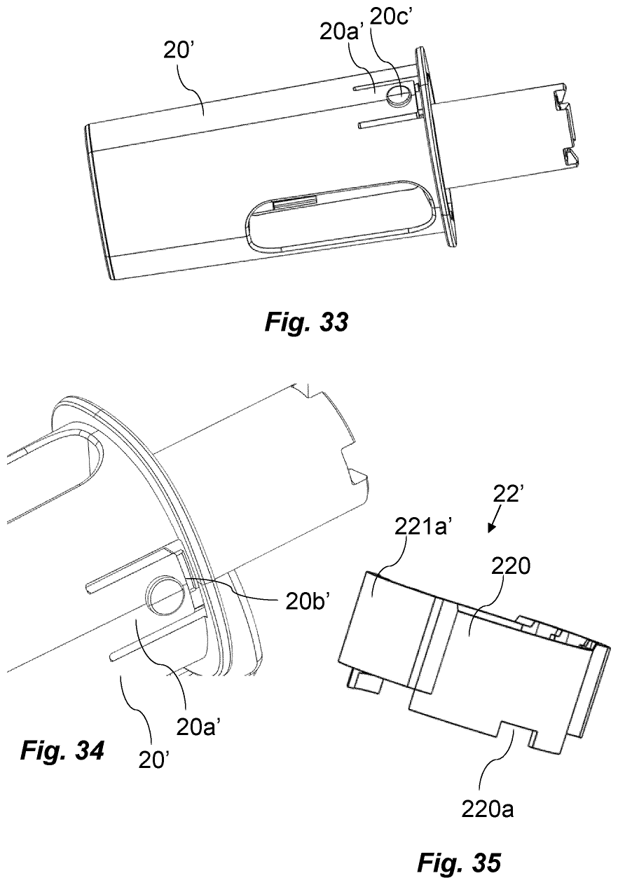

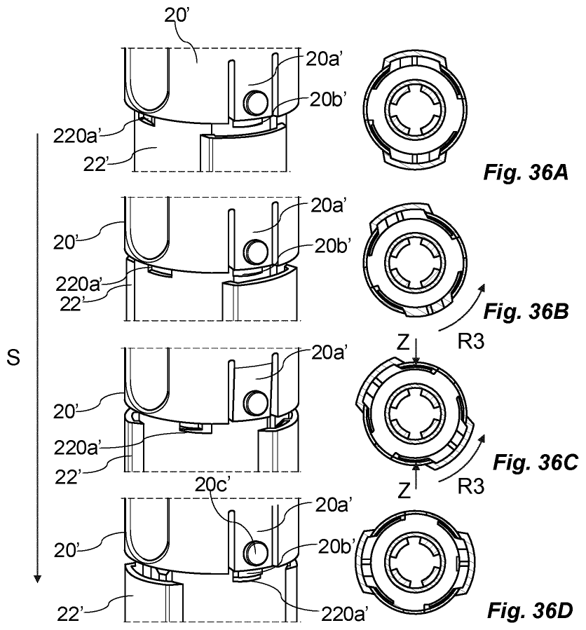

The tubular outer cap 30; 30′; 30″ comprises a plurality of engaging members 301; 301′. In a preferred example, the plurality of engaging members 301; 301′ of the tubular outer cap 30; 30′; 30″ is a plurality of protrusions extending radially inwardly relative to the longitudinal axis L from an inner surface of the tubular outer cap 30; 30′; 30″. The clutch member 32 comprises a plurality of counter engaging members 33, as shown in FIG. 24. The plurality of counter engaging members 33 can be a plurality of protrusions or slots facing radially outwardly relative to the longitudinal axis L from an outer surface of clutch member 32. The plurality of engaging members 301; 301′ is adjacent to the plurality of counter engaging members 33 when the clutch biasing member is in the tensioned position, as shown in FIG. 25B and FIG. 26B.

In a preferred example, the clutch member 32; 32′ comprises a proximal connector 321; 321′ and a distal connector 322; 322′. The proximal connector and/or distal connector can be ring-shaped or formed by multiple bracket-shaped elements. The clutch biasing member 320; 320′ is longitudinally positioned between the proximal connector 321; 321′ and the distal connector 322; 322′. The distal connector 322; 322′ is adjacent to the proximal end of the front shield 210; 210″ of the delivery member guard 21; 21′; 21″ and the proximal connector 321; 321′ is releasably adjacent to a distally directed surface on the inner surface of the tubular outer cap 30; 30′; 30″. In another example, the plurality of counter engaging members 33 of the clutch member 32; 32′ is arranged on an outer surface of the distal connector 322; 322′, as an example shown in FIG. 24; alternatively, the plurality of counter engaging members of the clutch member can be arranged on an outer surface of the proximal connector. In another example, the plurality of counter engaging members 33 comprises a flexible arm 331 extending partially around the longitudinal axis L, as shown in FIG. 24. In this example, when the plurality of engaging members 301 of the tubular outer cap 30 is adjacent to the plurality of counter engaging members 33 of the clutch member 32, and the outer cap is rotated in a predetermined direction, a tactile and/or audible indication can be provided to the end-user. The indication may be defined as a wrong manipulation indication or a correct manipulation indication, dependent on the design.

The tubular outer cap 30; 30′; 30″ is rotatable relative to the inner cap 31, so that only when the plurality of engaging members 301; 301′ of the tubular outer cap 30; 30′; 30″ is adjacent to the plurality of counter engaging members 33 of the clutch member 32; 32′, a rotation of the tubular outer cap 30; 30′; 30″ can be transferred to a rotation of the inner cap 31.

As shown in FIG. 25A, when the clutch biasing member 320 is in the relaxed position, the plurality of engaging members 301 of the tubular outer cap 30 is spaced apart from the plurality of counter engaging members 33 of the clutch member 32. When the delivery member guard 21; 21′ moves from its distal position to its proximal position, the delivery member guard 21; 21′ will move the clutch biasing member 320 to the tensioned position of the clutch biasing member 320. Therefore, the plurality of engaging members 301 of the tubular outer cap 30 is adjacent to the plurality of counter engaging members 33 of the clutch member 32.

It should be noted that, in the above-mentioned example, the delivery member guard 21; 21′ reaches to its proximal position when the tubular outer cap 30; 30′ is still attached to the sub-assembly. On the other hand, the delivery member guard 21; 21′ can also be arranged that the delivery member guard only reaches to its proximal position when the tubular outer cap 30; 30′; 30″ is completely detached from the sub-assembly. The latter example may be more suitable for the sub-assembly being arranged that the user is able to reversely rotate lock member from the unlocked position to the locked position.

As mentioned above, the sub-assembly of the invention is arranged such that the lock member 22; 22′; 22″ will release the delivery member guard 21; 21′; 21″ from locking at the distal position of the delivery member guard 21; 21′; 21″ only when the end-user has properly assembled the cassette unit 2 to a drive unit 1 of a medicament delivery device. Therefore, when the cassette unit 2 comprises the preassembled medicament delivery member set 3; 3′; 3″ as mentioned, the end-user can detach the outer cap 30; 30′; 30″ and the inner cap 31 only when the cassette unit 2 has been properly assembled to the drive unit 1. In a preferred example, the clutch member 32; 32′ can be detached from the cassette unit 2 together with the detachment of the inner cap 31 and the outer cap 30; 30′; 30″ due to the engagement between the outer surface of the inner cap 31 and the inner surface of the clutch member 32; 32′.

In one example where the delivery member guard 21″ can also be arranged that the delivery member guard 21″ only reaches to its proximal position when the tubular outer cap 30″ is completely detached from the sub-assembly.

In this example, the ledge 222″ of the lock member 22′ can be either the helical ledge or the circumferential ledge as mentioned above. A cut-out 222a″ is arranged in the ledge 222″, as shown in FIGS. 31-32B. Therefore, when the end-user attaches the cassette unit 2 to the drive unit 1, the protrusion 214″ of the delivery member guard 21″ moves along the ledge 222″. When the protrusion 214″ disengages with the ledge 222″, the protrusion 214″ will move into the cut-out 222a″ together with the delivery member guard 21″ that moves from the distal position to the proximal position. When the protrusion 214″ moves into the cut-out 222a″, the rotation between the delivery member guard 21″ and the lock member 22″ is blocked.

Therefore, when the delivery member guard 21″ moves to the proximal position, the delivery member guard 21″ will move the clutch biasing member 320′ to the tensioned position of the clutch biasing member 320′. Therefore, the protrusion 214″ will move into the cut-out 222a″. As the relative rotation between the delivery member guard 21″ and the lock member 22″ is blocked, and also the delivery member guard 21″ cannot rotate relative to the body 20 (as mentioned above), the end-user cannot detach the cassette unit 2 from the drive unit 1 before the outer cap 30″ is removed. When the outer cap 30″ is removed, the delivery member guard 21″ moves into the proximal position, the protrusion 214″ proximally moves out from the cut-out 222a″. Thus, the end-user can remove the cassette unit 2 from the drive unit 1.

In another example, the preassembled medicament delivery member set 3″ further comprises an adapter 34, as shown in FIG. 28. The adapter 34 comprises an adapter body 340. The adapter body 340 is rotationally and axially fixed to the inner cap 31. The adapter body 340 can be cylindrical as shown in FIG. 28, so that the medicament delivery device can be more compact. Alternatively, the adapter body 340 can be any suitable shape dependent on the design of the preassembled medicament delivery member set 3″. The adapter further comprises a track 341, the track 341 can be a recess/cut-out in the wall of the adapter body 340. In this example, the clutch member 32′ radially arranged between the tubular outer cap 30″ and the adapter body 340. In this example, the clutch member 32′ comprises a track follower 322b′ positioned within the track 341 of the adapter 34. The track follower 322b′ can be a protrusion extending from the inner surface of the distal connector 322′, as shown in FIG. 29.

As mentioned above, when the delivery member guard 21″ moves to the distal position, the delivery member guard 21″ will move the clutch biasing member 320 to the tensioned position of the clutch biasing member 320′. In the preferred example as shown in FIGS. 30A-30D, the delivery member guard 21″ is configured to move the distal connector 322′ of the clutch member 32′ in the distal direction relative to the inner cap 31 and the adapter 34. Therefore, the track follower 322b′ will be moved into the track 341 and will be moved along the track 341 of the adapter 34 by the front shield 210″ of the delivery member guard 21″. The track 341 comprises a circumferential section and a longitudinal section. In this example, when the delivery member guard 21″ moves to the proximal position, the delivery member guard 21″ moves the track follower 322b′ into the circumferential section of the track 341. If the end-user plans to remove the outer cap 30″, the end-user can rotate the outer cap 30″ together with the clutch element 32″, as the track follower 322b′ is in the circumferential section of the track 341, the inner cap 31 will not be rotated. When the track follower 322b′ aligns with the longitudinal section of the track 341, the delivery member guard 21″ will move the track follower 322b′ into the longitudinal section of the track 341, thus, when the end-user rotates the outer cap 30″ relative to the body 20, the outer cap 30″ can be removed, as mentioned above.

In this example, the protrusion 214″ of the delivery member guard 21″ is configured to position within the cut-out 222a″ when the track follower 322b′ is in the longitudinal section of the track 341. Therefore, the end-user will only be prevented from detaching the cassette unit 2 from the drive unit 1 when during the removal of the outer cap 30″.

The inventive concept has mainly been described above with reference to a few examples. However, as is readily appreciated by a person skilled in the art, other embodiments than the ones disclosed above are equally possible within the scope of the inventive concept, as defined by the appended claims.

1. A cassette unit sub-assembly for a medicament delivery device, the sub-assembly comprising:
a body (20) extending along a longitudinal axis (L) from a proximal end to a distal end;
a delivery member guard (21) coaxially attached to the body (20) and being axially movable relative to the body (20) along the longitudinal axis (L);
a lock member (22) attached to the body (20);
wherein the lock member (22) is movable relative to the delivery member guard (21) between a locked position and an unlocked position;
wherein the lock member (22) comprises a distally directed surface adjacent to a proximally directed surface of the delivery member guard (21) in the locked position; and wherein the distally directed surface of the lock member (22) is spaced apart in a circumferential direction relative to the longitudinal axis (L) from the proximally directed surface of the delivery member guard (21) in the unlocked position.

2. The cassette unit sub-assembly according to claim 1, wherein the lock member (22) is rotatable relative to the delivery member guard (21) around the longitudinal axis (L) between the locked position and the unlocked position.

3. The cassette unit sub-assembly according to claim 1, comprising a biasing member (23) arranged between a first distally directed surface of the delivery member guard (21) and a proximally directed surface of the body (20).

4. The cassette unit sub-assembly according to claim 1, wherein the delivery member guard (21) comprises a protrusion (214) radially extending relative to the longitudinal axis (L); and wherein the proximally directed surface of the delivery member guard (21) is a part of the protrusion (214).

5. The cassette unit sub-assembly according to claim 3, wherein the delivery member guard (21) comprises a ledge (212) radially extending relative to the longitudinal axis (L); and wherein the first distally directed surface of the delivery member guard (21) is defined by a part of the ledge (212).

6. The cassette unit sub-assembly according to claim 5, wherein the protrusion (214) extends radially away from the ledge (212).

7. The cassette unit sub-assembly according to claim 1, wherein the lock member (22) comprises a tubular body (220) enclosing a portion of the delivery member guard (21).

8. The cassette unit sub-assembly for a medicament delivery device according to claim 7, wherein the lock member (22) comprises a ledge (222; 222′) extending in a circumferential direction relative to the longitudinal axis (L) along a portion of an inner surface of the tubular body (220) of the lock member (22); and wherein the distally directed surface of the lock member (22) is a part of the ledge (222; 222′).

9. A cassette unit (2) for a medicament delivery device, the cassette unit (2) comprising the cassette unit sub-assembly according to claim 1, the cassette unit (2) comprising:
a delivery member cover assembly (3) fixed to the proximal end of the body (20) and being rotationally releasable relative to the body (20);
wherein the delivery member cover assembly (3) comprises:
a tubular outer cap (30);
an inner cap (31) at least partially surrounded by the tubular outer cap (30);
a clutch member (32) rotationally fixed to the inner cap (31) and positioned longitudinally between the tubular outer cap (30) and the delivery member guard (21);
wherein the clutch member (32) comprises a clutch biasing member (320), the clutch member (32) being axially movable relative to the outer cap (30) between a relaxed position and a tensioned position;
wherein the tubular outer cap (30) is axially fixed with and rotatable relative to the inner cap (31);
wherein the tubular outer cap (30) comprises a plurality of engaging members (301) and the clutch member comprises a plurality of counter engaging members (33); and wherein the plurality of engaging members (301) is adjacent with the plurality of counter engaging members (33) when the clutch biasing member (320) is in the tensioned position.

10. The cassette unit according to claim 9, wherein the plurality of engaging members (301) of the tubular outer cap (30) is a plurality of protrusions or slots; wherein the plurality of protrusions extends radially inwardly relative to the longitudinal axis from an inner surface of the tubular outer cap (30), or the plurality of slots faces radially outwardly relative to the longitudinal axis from an outer surface of the inner cap (31).

11. The cassette unit according to claim 10, wherein the plurality of counter engaging members (33) of the clutch member (32) is a plurality of counter protrusions (33); wherein the plurality of counter protrusions (33) extends radially outwardly relative to the longitudinal axis from an outer surface of the inner cap (31).

12. The cassette unit according to claim 9; wherein the clutch member (32) comprises a proximal connector (321) and a distal connector (322); wherein the clutch biasing member (320) is longitudinally positioned between the proximal connector (321) and the distal connector (322); and wherein the distal connector (322) is adjacent to a proximal end of the delivery member guard (21) and the proximal connector (321) is releasably adjacent to a distally directed surface on the inner surface of the tubular outer cap (30).

13. The cassette unit according to claim 12, wherein the plurality of counter engaging members (33) of the clutch member (32) is arranged on an outer surface of the distal connector (322).

14. The cassette unit according to claim 9, wherein the plurality of counter engaging members (33) comprises a flexible arm (331) extending partially around the longitudinal axis (L).

15. A medicament delivery device comprising the cassette unit (2) according to claim 9 and a reusable drive assembly (1);
wherein the reusable drive assembly (1) comprises a tubular housing extending along the longitudinal axis (L) from a proximal end and a distal end; and
wherein the cassette unit (2) is releasably attached to the reusable drive assembly (1) through a bayonet connection (11) on an inner surface on the proximal end of the reusable drive assembly (1) and on an outer surface on the distal end of the body of the cassette unit (2).
